(19) United States
(12) Patent Application Publication
(10) Pub. No.: US 2013/0123126 A1
(43) Pub. Date: May 16, 2013

(54) SELECTION AND USE OF HOST CELLS FOR PRODUCTION OF GLYCOPROTEINS

(71) Applicants: Brian Edward Collins, Arlington, MA (US); Jay Duffner, Shirley, MA (US); Victor Farutin, Watertown, MA (US); Naveen Bhatnagar, Framingham, MA (US); Lakshmanan Thiruneelakantapillai, Boston, MA (US); Carlos J. Bosques, Arlington, MA (US); Ganesh Kaundinya, Bedford, MA (US)

(72) Inventors: Brian Edward Collins, Arlington, MA (US); Jay Duffner, Shirley, MA (US); Victor Farutin, Watertown, MA (US); Naveen Bhatnagar, Framingham, MA (US); Lakshmanan Thiruneelakantapillai, Boston, MA (US); Carlos J. Bosques, Arlington, MA (US); Ganesh Kaundinya, Bedford, MA (US)

(73) Assignee: MOMENTA PHARMACEUTICALS, INC., Cambridge, MA (US)

(21) Appl. No.: 13/637,972

(22) PCT Filed: Apr. 7, 2011
(86) PCT No.: PCT/US11/31641

§ 371 Date: Jan. 30, 2013

Related U.S. Application Data
(60) Provisional application No. 61/321,863, filed on Apr. 7, 2010.

(51) Int. Cl. C12Q 1/04; G06F 19/00; C12Q 1/68

(52) U.S. Cl. CPC C12Q 1/04; C12Q 1/6809; G06F 19/32
USPC 506/9; 506/10; 707/705

(57) ABSTRACT

A method of making a glycoprotein having a selected glycostructure.

This application claims priority to U.S. Application Ser. No. 61/321,863, filed on Apr. 7, 2010. The disclosure of the prior application is considered part of (and is incorporated by reference in) the disclosure of this application.

The invention is directed to methods of selecting host cells for the production of glycoproteins, host cells, and other related methods, cells and glycoproteins.

### BACKGROUND

A typical glycoprotein product differs substantially in terms of complexity from a typical small molecule drug. The sugar structures attached to the amino acid backbone of a glycoprotein can vary structurally in many ways including, sequence, branching, sugar content, and heterogeneity. Thus, glycoprotein products can be complex heterogeneous mixtures of many structurally diverse molecules which themselves have complex glycan structures. Glycosylation adds not only to the molecule's structural complexity but affects or conditions many of a glycoprotein's biological and clinical attributes.

### SUMMARY

The appearance of post-translational modifications, e.g., glycostructures, glycan complement, glycan component, on proteins, is the result of an extremely complex interplay of many factors. Methods described herein rely, in part, on multi-observational analysis of the character of post-translational modifications, e.g., glycostructures, glycan complement, glycan component, on proteins made from selected cell populations. The methods allow comparisons of the ability of different cell populations in terms of their ability to confer complicated post-translational modifications, e.g., glycostructures, glycan complement, glycan component, on the proteins they make. The cell population quality attribute profiles provide for surprisingly robust distinctions between cell populations, even for very similar cell lines. Accordingly, the methods described herein can be used to select an appropriate host cell for production of a target glycoprotein (e.g., for production of a biosimilar or biogeneric product of a marketed biologic therapeutic glycoprotein), e.g., the methods described herein can be used to identify and/or select a host cell for production of a biosimilar or biogeneric product that best matches the glycosylation properties of the host cell in which the marketed biologic therapeutic glycoprotein was produced, e.g., in cases where the host cell population in which the marketed biologic therapeutic glycoprotein was produced is unknown to the maker of the biosimilar or biogeneric product. In aspects, an appropriate host cell population for production of a target glycoprotein is selected using methods described herein.

In one aspect, the invention features, a method of making a glycoprotein having a selected post-translational modification (e.g., a selected glycostructure, glycan complement, glycan component, e.g., with a selected glycan structure), or providing or selecting a cell population, e.g., a CHO cell population, e.g., for use in making a glycoprotein having a selected post-translational modification (e.g., a selected glycostructure, glycan complement, glycan component, e.g., with a selected glycan structure). The method comprises:

(a) acquiring, directly or indirectly, the identity of a cell population for the production of said glycoprotein, wherein the identity is acquired or determined by a method described herein, e.g., by
(i) acquiring, for each of a plurality of isolates or aliquots of a first cell population, a value which is expressed in terms of a post-translational modification, which value is a function of a plurality of distinct observations (e.g., the level of expression of a plurality of different genes, or the level of expression of a plurality of different glycostructures, glycan structures, glycan components, or combinations thereof) to provide a set of values for said first cell population;
(ii) acquiring, for each of a plurality of isolates or aliquots of a second cell population, a value which is expressed in terms of a post-translational modification, which value is a function of a plurality of distinct observations (e.g., the level of expression of a plurality of different genes, or the level of expression of a plurality of different glycostructures, glycan structures, glycan components, or combinations thereof) to provide a set of values for said second cell population;
(iii) comparing a value for a selected post-translational modification (e.g., glycostructure, glycan complement, glycan component, or combinations thereof) with the set of values for said first cell population and with the set of values for said second cell population; and
(iv) responsive to said comparison, selecting said first or second cell population.

In one embodiment, the method is a method of providing or selecting a cell population, e.g., a CHO cell population, e.g., for use in making a glycoprotein having a selected post-translational modification (e.g., a selected glycostructure, glycan complement, glycan component, e.g., with a selected glycan structure) and the method further comprises (b) culturing said selected cell population.

In an embodiment, the method is a method of making a glycoprotein having a selected post-translational modification (e.g., a selected glycostructure, glycan complement, glycan component, e.g., with a selected glycan structure) and, and the method further comprises (b) making a glycoprotein having a selected post-translational modification (e.g., glycostructure, glycan complement or glycan component, e.g., with a selected glycan structure) in said selected cell population.

In one embodiment, the method can further comprise genetically modifying the identified cell population to express said glycoprotein, e.g., introducing a nucleic acid that encodes all or part of said glycoprotein into said identified cell population prior to step (b).

In an embodiment, a set of values is acquired for a plurality, e.g., at least 3, 4, 5, 6, 7, 8, 9, 10 of cell populations.

In an embodiment, each of said cell populations in said plurality is from the same species, tissue, and cell type, though in embodiments they may differ by naturally acquired or intentionally induced mutations.

In some embodiments, each of the cell populations in said plurality is derived from a different cell line.

In an embodiment, each of the cell populations in said plurality is derived from a different single cell clone of a specific cell line.

In an embodiment, each of said cell populations in said plurality is a closely related cell population.

In an embodiment, each cell population of the cell populations shares a common ancestor cell wherein the ancestor cell was not part of an organism, e.g., the ancestor cell was a cultured cell or a founder cell of a cell line. Typically, the common ancestor cell is a cell, e.g., a cultured cell, that has been removed from a multicellular organism, e.g., an insect or animal, e.g., a mammal or primate, excluding as a common ancestor cell, precursor cells of the animal or ancestors of the animal from which the common ancestor cell is taken.

In an embodiment, each of the cell populations is derived from a common ancestor cell and none of the cell populations of the plurality has an intentionally induced mutation that inactivates a gene encoding a protein which synthesizes attaches or modifies a glycan. In an embodiment, each of the cell populations of the plurality is derived from a common ancestor cell and none of the cell populations of the plurality has an intentionally induced inactivating mutation in a gene encoding a protein selected from: a glycosyltransferase (e.g., MGAT1 (GlcNAc T1), alpha mannosidase II, IIx, alpha mannosidase IB, alpha mannosidase IA, FucT1-9, glucosidase (e.g., GCS1, GANAB), a precursor to biosynthesis or localization or trafficking, GNE (e.g., glucosamine (UDP-N-acetyl)-2-epimerase/N-acetylmannosamine), Golgi UDP phosphatase, UDP-GlcNAc transporter, UAP-1 (UDP-N-acetylhexosamine pyrophosphorylase), PGM-3—phosphoglucomutase 3, NAGK—N-acetyl-D-glucosamine kinase, GNPNAT1—glucosamine-phosphate N-acetyltransferase 1, UGP-2—UDP-glucose pyrophosphorylase 2, UGDH—UDP-glucose 6-dehydrogenaseGAlK-1—Galactokinase-1, PGM-1—Phosphoglucomutase-1, GCK-glucokinase), a target to alter the localization or trafficking through the ER and golgi, e.g., a chaperone (BiP, SNARE, cpn, hsp), EDEM (ER degrading mannosidase-like protein), MANEA, mannose receptor. In an embodiment, each of the cell populations of the plurality is derived from a common ancestor cell and none of the cell populations of the plurality has an intentionally induced inactivating mutation that modulates the level of a glycan metabolite, e.g., a metabolite described herein.

In one embodiment, the cell populations are not derived from a Pro-5 cell line. In an embodiment, the cell populations are not modified (e.g., not chemically mutagenized) to be resistant to a lectin.

In an embodiment, the selected post-translational modification is a selected glycan complement or glycan component.

In an embodiment, the glycoprotein is a therapeutic biologic product, e.g., a therapeutic antibody, Fc-receptor fusion, hormone, cytokine. In one embodiment, the glycoprotein is a biosimilar or biogeneric version of a marketed therapeutic biologic product.

In one embodiment, the observations for each cell population include at least 2, 3, 4, 5, 6, 7, 8, 9, 10 or more observations of the expression levels of genes. In an embodiment, the observations for each cell population include at least 2, 3, 4, 5, 6, 7, 8, 9, 10 or more observations regarding the levels of a glycan metabolite.

In an embodiment, the method, e.g., (i)-(iv) comprises:
(i) acquiring a cell population quality attribute profile (a profile), comprising a set of answers, wherein an answer is expressed in terms of a post-translational modification and is the product of an operation on a plurality of observations, for each of a plurality of cell populations, said acquired profiles forming a plurality of distinct profiles;
(ii) acquiring the identity of a selected post-translational modification (e.g., glycostructure, glycan complement, glycan component, or combination thereof);
(iii) comparing the acquired profile with the identity acquired in (ii);
(iv) responsive to the comparison (e.g., when the acquired profile and the identity acquired in (ii) show a preselected relationship with one another, e.g., the former includes the latter), selecting one of the plurality of cell populations for production of the subject glycoprotein; selecting or providing the cell population to make said glycoprotein and/or making said glycoprotein having the selected post-translational modification (e.g., glycostructure) in said selected cell population. In some embodiments, the method further comprises introducing a nucleic acid that encodes all or part of said glycoprotein into said identified cell population.

In an embodiment, the identity of said cell population is directly acquired.

In an embodiment, the identity of said cell population is indirectly acquired.

In an embodiment, dimensionality of an answer is less than the dimensionality of the number of observations.

In an embodiment, the method comprises a manipulation that reduces the dimensionality of the answer, as compared with the number of observations.

In an embodiment, the comparison is made with answer′, wherein answer′ has at least one less dimension than the answer.

In an embodiment, the method comprises a manipulation that reduces the dimensionality of an answer′, as compared with an answer.

In some embodiments, an underlying observation is expressed in terms of glycan structure, glycostructure, glycan component or glycan complement. Such an embodiment can have one or more of the following properties:

the answers in said acquired profile are based on a first and second observation and said first observation is the level of a first post-translational modification, e.g., glycan structure, glycostructure, glycan component or glycan complement, and the second observation is the level of a second post-translational modification, e.g., glycan structure, glycostructure, glycan component or glycan complement;

the comparison comprises comparing the selected post-translational modification, e.g., glycostructure, with an dimensional representation of said plurality of profiles wherein the axis in each dimension represents a different aspect of glycostructure, glycan complement or glycan component, e.g., wherein the axis for a first dimension represents the level of glycan A and the axis for a second dimension represents the level of glycan B.

In some embodiments, an underlying observation is not expressed in terms of glycan structure and is expressed, e.g., in terms of the expression level of one or more genes. In such embodiments, the operation not only gives an answer but also puts the answer in terms of glycan structure. Such an embodiment can have one or more of the following properties:

the answers in said acquired profile are based on a first and second observation and at least one of said first and second observations are not expressed in terms of post-translational structure, e.g., glycostructure, but are expressed in terms of a parameter related to post-translational structure, e.g., glycostructure, and the operation provides an answer expressed in terms of post-translational structure, e.g., glycostructure, glycan complement or glycan component;

the answers in said acquired profile are based on a first and second observation and said first observation is the level of a first metabolite and the second observation is the level of a second metabolite;

the comparison comprises comparing the answer for the selected glycostructure, glycan complement or glycan component with an n dimensional depiction of said plurality of distinct acquired profiles wherein the axis in each dimension is correlated with a different aspect of glycostructure, glycan complement or glycan component, e.g., wherein the axis for a first dimension is correlated with the level of glycan A and the axis for a second dimension is correlated with the level of glycan B.

The method requires “acquiring” steps, e.g., acquiring a profile or acquiring the identity of a selected post-translational modification. Acquiring the method can include one of a number of elements.

In an embodiment acquiring a value comprises subjecting a sample to a process which results in a physical change in the sample or another substance, e.g., an analytical reagent or a device used in the analysis. Such methods comprise analytical methods, e.g., a method which include one or more of the following: separating a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment of other derivative thereof, e.g., by breaking or forming a covalent or non covalent bond, between a first and a second atom of the analyte or a reagent.

In other embodiments, e.g., in embodiments where the method includes the production of a glycoprotein, or culturing a cell, harvesting a glycoprotein or purifying a glycoprotein, or another step which results in a transformation of an entity used in the method, e.g., a cell, glycoprotein or reagent, the acquiring step may be a step that can be yielded without such a transformation, e.g., by inspection, comparing or receiving information from another party.

In an embodiment, acquiring a profile comprises performing chemical or physical analysis to determine the profile.

In an embodiment, acquiring a profile comprises receiving information regarding the profile from another party.

In an embodiment, acquiring the identity of a post-translational modification comprises performing a chemical or physical analysis to determine the identity.

In an embodiment, acquiring the identity of a post-translational modification comprises selecting the identity from a description of a drug, e.g., from a package insert.

In an embodiment, acquiring the identity of a post-translational modification comprises selecting the identity from a list or table.

In an embodiment, acquiring the identity of a post-translational modification comprises receiving information regarding the identity of the post-translational modification from another party.

As discussed elsewhere herein, an observation can be expressed in terms of glycan structure.

In an embodiment, an observation is the level of 4,4,1,0,0.

In an embodiment, an observation is the level of 4,4,1,1,0.

In an embodiment, an observation is the level of 4,5,1,0,0.

In an embodiment, an observation is the level of 4,5,1,1,0.

In an embodiment, an observation is the level of 4,5,1,2,0.

In an embodiment, an observation is the level of 5,5,1,0,0.

In an embodiment, an observation is the level of 5,6,1,0,0.

In an embodiment, an observation is the level of 5,6,1,1,0.

In an embodiment, an observation is the level of 5,6,1,2,0.

In an embodiment, an observation is the level of 5,6,1,3,0.

In an embodiment, an observation is the level of 6,6,1,1,0.

In an embodiment, an observation is the level of 6,6,1,2,0.

In an embodiment, an observation is the level of 6,7,1,1,0.

In an embodiment, an observation is the level of 6,7,1,2,0.

In an embodiment, an observation is the level of 6,7,1,3,0.

In an embodiment, an observation is the level of 6,7,1,4,0.

As discussed elsewhere herein, an observation can be expressed in terms other than of glycan structure, glycan complement or glycan component. In an embodiment, an observation is the level of gene expression.

In an embodiment, an observation is the level of expression of a glycosyltransferase.

In an embodiment, an observation is the level of expression of a gene involved in glycan biosynthesis.

In an embodiment, an observation is the level of a metabolite.

In an embodiment, an observation is the level of UMP.

In an embodiment, an observation is the level of GTP.

In an embodiment, an observation is the level of UDP-Gal.

In an embodiment, an observation is the level of GDP-Fuc.

As discussed elsewhere herein methods described herein can be used with a range of cell populations, e.g., different cell strains from a parental cell line or isolates from a parental cell strain.

In an embodiment, one of the cell populations of the plurality of cell populations is a CHO cell line.

In an embodiment, one of the cell populations of the plurality of cell populations is a CHO K1 cell line.

In an embodiment, one of the cell populations of the plurality of cell populations is a CHO S cell line.

In an embodiment, one of the cell populations of the plurality of cell populations is a DG44 cell line.

In an embodiment, one of the cell populations of the plurality of cell populations is a DHFR(−) cell line.

In an embodiment, one of the cell populations of the plurality of cell populations is a CHO GS cell line.

As discussed elsewhere in methods described herein, a number of types of operation are suitable for use in the methods.

In an embodiment, the operation is an arithmetic combination of a plurality of observations.

In an embodiment, the operation is a fit to a model of a plurality of observations.

In an embodiment, the model is a linear model.

In an embodiment, the operation comprises relating, e.g., associating, correlating or equating, values for observations derived from a source of information, e.g., a list, table, or database, e.g., publicly available database.

As discussed elsewhere in methods described herein, a number of types of answers are suitable for use in the methods.

In an embodiment, the answer is the product of an operation on the level of expression of a plurality of genes, e.g., wherein: at least one of the plurality of genes encodes a protein that forms the selected post-translational modification; at least one of the plurality of genes encodes a protein that reduces the level of the selected post-translational modification; the answer is the product of an operation on the levels of ST3GAL3 and ST3GAL4.

In another aspect, the invention features, a method of providing or selecting a cell population from a plurality of isolates of the same cell type, e.g., isolates from a CHO cell population, e.g., for use in making a glycoprotein having a selected post-translational modification (e.g., a selected glycostructure, glycan complement or glycan component, e.g., with a selected glycan structure). The method comprises:

(a) acquiring the identity of a selected post-translational modification (e.g., glycostructure, glycan complement, glycan component, e.g., with a selected glycan structure);

(b) acquiring an evaluation, e.g., by use of method described herein, the ability of each of said plurality of isolates of said cell type to produce said selected post-translation modification,

(c) selecting an isolate from said plurality of isolates,

(d) optionally culturing said selected cell population;

thereby providing a cell population.

In one embodiment, the method further comprises (b) culturing said selected cell population.

In an embodiment, the method further comprises (b) making a glycoprotein having a selected post-translational modification (e.g., glycostructure, glycan complement or glycan component, e.g., with a selected glycan structure) in said selected cell population.

In one embodiment, the method can further comprise genetically modifying the identified cell population to express said glycoprotein, e.g., introducing a nucleic acid that encodes all or part of said glycoprotein into said identified cell population prior to step (b).

In an embodiment, a set of values is acquired for a plurality, e.g., at least 3, 4, 5, 6, 7, 8, 9, 10 of cell populations.

In an embodiment, each of said cell populations in said plurality is from the same species, tissue, and cell type, though in embodiments they may differ by naturally acquired or intentionally induced mutations.

In some embodiments, each of the cell populations in said plurality is derived from a different cell line, different cell strain, or different clone.

In an embodiment, each of the cell populations in said plurality is derived from a different single cell clone of a specific cell line.

In an embodiment, each of said cell populations in said plurality is a closely related cell population.

In an embodiment, each cell population of the cell populations shares a common ancestor cell wherein the ancestor cell was not part of an organism, e.g., the ancestor cell was a cultured cell or a founder cell of a cell line. Typically, the common ancestor cell is a cell, e.g., a cultured cell, that has been removed from a multicellular organism, e.g., an insect or animal, e.g., a mammal or primate, excluding as a common ancestor cell, precursor cells of the animal or ancestors of the animal from which the common ancestor cell is taken.

In an embodiment, each of the cell populations is derived from a common ancestor cell and none of the cell populations of the plurality has an intentionally induced mutation that inactivates a gene encoding a protein which synthesizes, attaches or modifies a glycan. In an embodiment, each of the cell populations of the plurality is derived from a common ancestor cell and none of the cell populations of the plurality has an intentionally induced inactivating mutation in a gene encoding a protein selected from: a glycosyltransferase (e.g., MGAT1 (GlcNAc T1), alpha mannosidase II, IIx, alpha mannosidase IB, alpha mannosidase IA, FucT1-9, glucosidase (e.g., GCS1, GANAB), a precursor to biosynthesis or localization or trafficking, GNE (e.g., glucosamine (UDP-N-acetyl)-2-epimerase/N-acetylmannosamine), Golgi UDP phosphatase, UDP-GlcNAc transporter, UAP-1 (UDP-N-acetylhexosamine pyrophosphorylase), PGM-3—phosphoglucomutase 3, NAGK—N-acetyl-D-glucosamine kinase, GNPNAT1—glucosamine-phosphate N-acetyltransferase 1, UGP-2—UDP-glucose pyrophosphorylase 2, UGDH—UDP-glucose 6-dehydrogenaseGAlK—1—Galactokinase-1, PGM-1—Phosphoglucomutase-1, GCK—glucokinase), a target to alter the localization or trafficking through the ER and golgi, e.g., a chaperone (BiP, SNARE, cpn, hsp), EDEM (ER degrading mannosidase-like protein), MANEA, mannose receptor. In an embodiment, each of the cell populations of the plurality is derived from a common ancestor cell and none of the cell populations of the plurality has an intentionally induced inactivating mutation that modulates the level of a glycan metabolite, e.g., a metabolite described herein.

In one embodiment, the cell populations are not derived from a Pro-5 cell line. In an embodiment, the cell populations are not modified (e.g., not chemically mutagenized) to be resistant to a lectin.

In an embodiment, the selected post-translational modification is a selected glycan complement or glycan component.

In an embodiment, the glycoprotein is a therapeutic biologic product, e.g., a therapeutic antibody, Fc-receptor fusion, hormone, cytokine. In one embodiment, the glycoprotein is a biosimilar or biogeneric version of a marketed therapeutic biologic product.

In one embodiment, the observations for each cell population include at least 2, 3, 4, 5, 6, 7, 8, 9, 10 or more observations of the expression levels of genes. In an embodiment, the observations for each cell population include at least 2, 3, 4, 5, 6, 7, 8, 9, 10 or more observations regarding the levels of a glycan metabolite.

In an embodiment, the method, e.g., (i)-(iv) comprises:
(i) acquiring a cell population quality attribute profile (a profile), comprising a set of answers, wherein an answer is expressed in terms of a post-translational modification and is the product of an operation on a plurality of observations, for each of a plurality of cell populations, said acquired profiles forming a plurality of distinct profiles;
(ii) acquiring the identity of a selected post-translational modification (e.g., glycostructure, glycan complement or glycan component);
(iii) comparing the acquired profile with the identity acquired in (ii);
(iv) responsive to the comparison (e.g., when the acquired profile and the identity acquired in (ii) show a preselected relationship with one another, e.g., the former includes the latter), selecting one of the plurality of cell populations for production of the subject glycoprotein; selecting or providing the cell population to make said glycoprotein and/or making said glycoprotein having the selected post-translational modification (e.g., glycostructure) in said selected cell population. In some embodiments, the method further comprises genetically modifying the identified cell population to express said glycoprotein, e.g., introducing a nucleic acid that encodes all or part of said glycoprotein into said identified cell population.

In an embodiment, the identity of said cell population is directly acquired.

In an embodiment, the identity of said cell population is indirectly acquired.

In an embodiment, dimensionality of an answer is less than the dimensionality of the number of observations.

In an embodiment, the method comprises a manipulation that reduces the dimensionality of the answer, as compared with the number of observations.

In an embodiment, the comparison is made with answer′, wherein answer′ has at least one less dimension than the answer.

In an embodiment, the method comprises a manipulation that reduces the dimensionality of an answer′, as compared with an answer.

In some embodiments, an underlying observation is expressed in terms of glycan structure, glycostructure, glycan component or glycan complement. Such an embodiment can have one or more of the following properties:

the answers in said acquired profile are based on a first and second observation and said first observation is the level of a first post-translational modification, e.g., glycan structure, glycostructure, glycan component or glycan complement, and the second observation is the level of a second post-translational modification, e.g., glycan structure, glycostructure, glycan component or glycan complement;

the comparison comprises comparing the selected post-translational modification, e.g., glycostructure, with an dimensional representation of said plurality of profiles wherein the axis in each dimension represents a different aspect of glycostructure, glycan complement or glycan component, e.g., wherein the axis for a first dimension represents the level of glycan A and the axis for a second dimension represents the level of glycan B.

In some embodiments, an underlying observation is not expressed in terms of glycan structure and is expressed, e.g., in terms of the expression level of one or more genes. In such embodiments, the operation not only gives an answer but also puts the answer in terms of glycan structure. Such an embodiment can have one or more of the following properties:

the answers in said acquired profile are based on a first and second observation and at least one of said first and second observations are not expressed in terms of post-translational structure, e.g., glycostructure, but are expressed in terms of a parameter related to post-translational structure, e.g., glycostructure, and the operation provides an answer expressed in terms of post-translational structure, e.g., glycostructure, glycan complement or glycan component;

the answers in said acquired profile are based on a first and second observation and said first observation is the level of a first metabolite and the second observation is the level of a second metabolite;

the comparison comprises comparing the answer for the selected glycostructure, glycan complement or glycan component with an n dimensional depiction of said plurality of distinct acquired profiles wherein the axis in each dimension is correlated with a different aspect of glycostructure, glycan complement or glycan component, e.g., wherein the axis for a first dimension is correlated with the level of glycan A and the axis for a second dimension is correlated with the level of glycan B.

The method requires “acquiring” steps, e.g., acquiring a profile or acquiring the identity of a selected post-translational modification. Acquiring the method can include one of a number of elements.

In an embodiment, acquiring a value comprises subjecting a sample to a process which results in a physical change in the sample or another substance, e.g., an analytical reagent or a device used in the analysis. Such methods comprise analytical methods, e.g., a method which include one or more of the following: separating a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment of other derivative thereof, e.g., by breaking or forming a covalent or non covalent bond, between a first and a second atom of the analyte or a reagent.

In other embodiments, e.g., in embodiments where the method includes the production of a glycoprotein, or culturing a cell, harvesting a glycoprotein or purifying a glycoprotein, or another step which results in a transformation of an entity used in the method, e.g., a cell, glycoprotein or reagent, the acquiring step may be a step that can be yielded without such a transformation, e.g., by inspection, comparing or receiving information from another party.

In an embodiment, acquiring a profile comprises performing chemical or physical analysis to determine the profile.

In an embodiment, acquiring a profile comprises receiving information regarding the profile from another party.

In an embodiment, acquiring the identity of a post-translational modification comprises performing a chemical or physical analysis to determine the identity.

In an embodiment, acquiring the identity of a post-translational modification comprises selecting the identity from a description of a drug, e.g., from a package insert.

In an embodiment, acquiring the identity of a post-translational modification comprises selecting the identity from a list or table.

In an embodiment, acquiring the identity of a post-translational modification comprises receiving information regarding the identity of the post-translational modification from another party.

As discussed elsewhere herein, an observation can be expressed in terms of glycan structure.

In an embodiment, an observation is the level of 4,4,1,0,0.

In an embodiment, an observation is the level of 4,4,1,1,0.

In an embodiment, an observation is the level of 4,5,1,0,0.

In an embodiment, an observation is the level of 4,5,1,1,0.

In an embodiment, an observation is the level of 4,5,1,2,0.

In an embodiment, an observation is the level of 5,5,1,0,0.

In an embodiment, an observation is the level of 5,6,1,0,0.

In an embodiment, an observation is the level of 5,6,1,1,0.

In an embodiment, an observation is the level of 5,6,1,2,0.

In an embodiment, an observation is the level of 5,6,1,3,0.

In an embodiment, an observation is the level of 6,6,1,1,0.

In an embodiment, an observation is the level of 6,6,1,2,0.

In an embodiment, an observation is the level of 6,7,1,1,0.

In an embodiment, an observation is the level of 6,7,1,2,0.

In an embodiment, an observation is the level of 6,7,1,3,0.

In an embodiment, an observation is the level of 6,7,1,4,0.

As discussed elsewhere herein, an observation can be expressed in terms other than of glycan structure, glycan complement or glycan component. In an embodiment, an observation is the level of gene expression.

In an embodiment, an observation is the level of expression of a glycosyltransferase.

In an embodiment, an observation is the level of expression of a gene involved in glycan biosynthesis.

In an embodiment, an observation is the level of a metabolite.

In an embodiment, an observation is the level of UMP.

In an embodiment, an observation is the level of GTP.

In an embodiment, an observation is the level of UDP-Gal.

In an embodiment, an observation is the level of GDP-Fuc.

As discussed elsewhere herein methods described herein can be used with a range of cell populations, e.g., different cell strains from a parental cell line or different isolates from a parental cell strain.

In an embodiment, one of the cell populations of the plurality of cell populations is a CHO cell line.

In an embodiment, one of the cell populations of the plurality of cell populations is a CHO K1 cell line.

In an embodiment, one of the cell populations of the plurality of cell populations is a CHO S cell line.

In an embodiment, one of the cell populations of the plurality of cell populations is a DG44 cell line.

In an embodiment, one of the cell populations of the plurality of cell populations is a DHFR(−) cell line.

In an embodiment, one of the cell populations of the plurality of cell populations is a CHO GS cell line.

As discussed elsewhere herein methods described herein a number of types of operation are suitable for use in the methods.

In an embodiment, the operation is an arithmetic combination of a plurality of observations.

In an embodiment, the operation is a fit to a model of a plurality of observations.

In an embodiment, the model is a linear model.

In an embodiment, the operation comprises relating, e.g., associating, correlating or equating, values for observations derived from a source of information, e.g., a list, table, or database, e.g., publicly available database.

As discussed elsewhere herein methods described herein a number of types of answers are suitable for use in the methods.

In an embodiment, the answer is the product of an operation on the level of expression of a plurality of genes, e.g., wherein: at least one of the plurality of genes encodes a protein that forms the selected post-translational modification; at least one of the plurality of genes encodes a protein that reduces the level of the selected post-translational modification; the answer is the product of an operation on the levels of ST3GAL3 and ST3GAL4.

In another aspect, the invention features, a method of selecting or evaluating a cell, e.g., for use in making a glycoprotein having a selected post-translational modification. The method comprises:

(a) acquiring, directly or indirectly, the identity of a cell population for the production of said glycoprotein, wherein the identity is determined by
(i) acquiring, for each of for each of a plurality of isolates or aliquots of a first cell population, a value which is expressed in terms of a post-translational modification, which value is a function of a plurality of distinct observations (e.g., the level of expression of a plurality of different genes, or the level of expression of a plurality of different glycostructure, glycan complement or glycan component, e.g., with a selected glycan structure) to proved a set of values for said first cell population;
(ii) acquiring, for each of for each of a plurality of isolates or aliquots of a second cell population, a value which is expressed in terms of a post-translational modification, which value is a function of a plurality of distinct observations to proved a set of values for said second cell population;
(iii) comparing a value for a selected post-translational modification (e.g., glycostructure, glycan complement or glycan component, e.g., with a selected glycan structure) with the set of values for said first cell population and with the set of values for said second cell population; and
(iv) responsive to said comparison, selecting said first or second cell population;

to thereby select or evaluate said cell, wherein:
(1) step (i and/or ii) comprises growing a cell population, performing a chemical or physical analysis to provide an answer, e.g., chemical or physical analysis to provide an observation.

In an embodiment, the method, e.g., (i)-(iv) comprises:
(i) acquiring a cell population quality attribute profile comprising a set of answers, wherein an answer is expressed in terms of a post-translational modification and is the product of an operation on a plurality of observations, for each of a plurality of cell populations, said acquired profiles forming a plurality of distinct profiles;
(ii) acquiring the identity of a selected post-translational modification (e.g., glycostructure, glycan complement or glycan component);
(iii) comparing the acquired profile with the identity acquired in (b);
(iv) responsive to the comparison (e.g., when the acquired profile and the identity acquired in (b) show a preselected relationship with one another, e.g., the former includes the latter), selecting one of the plurality of cell populations for production of the subject glycoprotein,
to thereby select or evaluate said cell, wherein:
   step (i) comprises growing a cell population, performing a chemical or physical analysis to provide an answer, e.g., chemical or physical analysis to provide an observation;
   step (ii) comprises performing a chemical or physical analysis to provide said identity;
   step (iii) comprises providing a representation of the profile as an n-dimensional space and comparing the identity with said space; or optionally, the method further comprises culturing said selected cell.

As discussed elsewhere herein, an observation can be expressed in terms of glycostructure, glycan complement or glycan component, e.g., with a selected glycan structure, e.g., a glycan structure disclosed herein.

As discussed elsewhere herein, an observation can be expressed in terms other than glycostructure, glycan complement or glycan component, e.g., with a selected glycan structure, e.g., the level of gene expression, e.g., a gene discussed herein, or a metabolite, e.g., a metabolite discussed herein.

As discussed elsewhere in methods described herein can be used with a range of cell populations, e.g., a CHO or other cell population described herein.

As discussed elsewhere in methods described herein a number of types of operation are suitable for use in the methods, e.g., operations discussed herein, e.g., an arithmetic combination or linear model.

As discussed elsewhere in methods described herein a number of types of answers are suitable for use in the methods, e.g., answer described herein, e.g., an answer which is the product of an operation on the level of expression of a plurality of genes.

As discussed elsewhere herein, the types of answers and/or observations can be the level of expression of a gene or genes described herein.

In another aspect, the invention features, a method of providing a population of cells, e.g., for use in making a glycoprotein having a selected post-translational modification. The method comprises:

(a) acquiring a cell population quality attribute profile comprising a set of answers, wherein an answer is expressed in terms of a post-translational modification and is the product of an operation on a plurality of observations, for each of a plurality of cell populations, said acquired profiles forming a plurality of distinct profiles;

(b) acquiring the identity of a selected post-translational modification (e.g., glycostructure, glycan complement or glycan component, e.g., with a selected glycan structure);

(c) comparing the acquired profile with the identity acquired in (b);

(d) responsive to the comparison (e.g., when the acquired profile and the identity acquired in (b) show a preselected relationship with one another, e.g., the former includes the latter), selecting one of the plurality of cell populations for production of the subject glycoprotein; and

(e) culturing said selected cell population to provide said population.

As discussed elsewhere herein a method can require one or more “acquiring” steps, e.g., acquiring a profile or acquiring the identity of a selected post-translational modification. In an embodiment acquiring a value comprises subjecting a sample to a process which results in a physical change in the sample or another substance, e.g., an analytical reagent or a device used in the analysis, e.g., such an analysis described herein. In other embodiments, e.g., in embodiments where the method includes the production of a glycoprotein, or culturing a cell, harvesting a glycoprotein or purifying a glycoprotein, or another step which results in a transformation of an entity used in the method, e.g., a cell, glycoprotein or reagent, the acquiring step may be a step that can be yielded without such a transformation, e.g., by inspection, comparing or receiving information from another party.

As discussed elsewhere herein, an observation can be expressed in terms of glycostructure, glycan complement or glycan component, e.g., with a selected glycan structure, e.g., a glycan structure disclosed herein.

As discussed elsewhere herein, an observation can be expressed in terms other than glycan structure, e.g., the level of gene expression, e.g., a gene discussed herein, or a metabolite, e.g., a metabolite discussed herein.

As discussed elsewhere in methods described herein can be used with a range of cell populations, e.g., a CHO or other cell population described herein.

As discussed elsewhere in methods described herein a number of types of operation are suitable for use in the methods, e.g., operations discussed herein, e.g., an arithmetic combination or linear model.

As discussed elsewhere in methods described herein a number of types of answers are suitable for use in the methods, e.g., answer described herein, e.g., an answer which is the product of an operation on the level of expression of a plurality of genes.

As discussed elsewhere herein, the types of answers and/or observations can be the level of expression of a gene or genes described herein.

In another aspect, the invention features, a method of monitoring a production process for making a glycoprotein having a selected post-translational modification. The method comprises:

(a) acquiring, for each of for each of a plurality of isolates or aliquots of a first cell population, a value which is expressed in terms of a post-translational modification, which value is a function of a plurality of distinct observations (e.g., the level of expression of a plurality of different genes, or the level of expression of a plurality of different glycostructure, glycan complement or glycan component, e.g., with a selected glycan structure) to proved a set of values for said first cell population;

(b) comparing a value for a selected post-translational modification (e.g., glycostructure, glycan complement or glycan component, e.g., with a selected glycan structure) with the set of values for said first cell population; and

(c) if the comparison shows a first preselected relationship with the set of values, e.g., the set of values includes the identity, pursuing a first option, e.g., continuing culture; and if the comparison shows a second preselected relationship with the set of value, e.g., the set of values does not include the identity, pursuing a second option, e.g., ceasing current culture conditions or culturing under a new set of conditions.

In an embodiment, the method comprises:

(a) acquiring a cell population quality attribute profile comprising a set of answers, wherein an answer is expressed in terms of a post-translational modification and is the product of an operation on a plurality of observations, for an aliquot of production cells;

(b) comparing the identity of a selected post-translational modification (e.g., glycostructure) with said profile;

(c) if the comparison shows a first preselected relationship with the profile, e.g., the profile includes the identity, pursuing a first option, e.g., continuing culture; and if the comparison shows a second preselected relationship with the profile, e.g., the profile does not include the identity, pursuing a second option, e.g., ceasing current culture conditions or culturing under a new set of conditions.

In one embodiment, the selected glycan component and/or glycan complement is a glycan component and/or glycan complement of a biologic therapeutic glycoprotein, e.g., a marketed biologic therapeutic glycoprotein, and if the profile includes the identity of the selected glycan component and/or glycan complement continuing to culture said CHO cells, e.g., to produce a biogeneric or biosimilar glycoprotein of said biologic therapeutic glycoprotein.

In one embodiment, the selected glycan component and/or glycan complement is a glycan component and/or glycan complement of a biologic therapeutic glycoprotein, e.g., a marketed biologic therapeutic glycoprotein, and if the profile does not include the identity of the selected glycan component and/or glycan complement pursing a second option, e.g., selecting a different CHO cell population that has a profile that includes the selected glycan component and/or glycan complement, e.g., to produce a biogeneric or biosimilar glycoprotein of said biologic therapeutic glycoprotein.

In one embodiment, the selected glycan component and/or glycan complement is a glycan component and/or glycan complement of a biologic therapeutic glycoprotein, e.g., a marketed biologic therapeutic glycoprotein, and if the profile includes the identity of the selected glycan component and/or glycan complement continuing to culture said CHO cells, e.g., to produce said biologic therapeutic glycoprotein.

In one embodiment, the selected glycan component and/or glycan complement is a glycan component and/or glycan complement of a biologic therapeutic glycoprotein, e.g., a marketed biologic therapeutic glycoprotein, and if the profile does not include the identity of the selected glycan component and/or glycan complement pursing a second option, e.g., ceasing current culture conditions or culturing under a new set of conditions, e.g., conditions that result in a profile that includes the identity of said selected glycan component and/or glycan complement, to produce said biologic therapeutic glycoprotein.

As discussed elsewhere herein a method can require one or more “acquiring” steps, e.g., acquiring a profile or acquiring the identity of a selected post-translational modification. In an embodiment acquiring a value comprises subjecting a sample to a process which results in a physical change in the sample or another substance, e.g., an analytical reagent or a device used in the analysis, e.g., such an analysis described herein. In other embodiments, e.g., in embodiments where the method includes the production of a glycoprotein, or culturing a cell, harvesting a glycoprotein or purifying a glycoprotein, or another step which results in a transformation of an entity used in the method, e.g., a cell, glycoprotein or reagent, the acquiring step may be a step that can be yielded without such a transformation, e.g., by inspection, comparing or receiving information from another party.

As discussed elsewhere herein, an observation can be expressed in terms of glycan structure, e.g., a glycan structure disclosed herein.

As discussed elsewhere herein, an observation can be expressed in terms other than glycan structure, e.g., the level of gene expression, e.g., a gene discussed herein, or a metabolite, e.g., a metabolite discussed herein.

As discussed elsewhere herein methods described herein can be used with a range of cell populations, e.g., a CHO or other cell population described herein.

As discussed elsewhere herein methods described herein a number of types of operation are suitable for use in the methods, e.g., operations discussed herein, e.g., an arithmetic combination or linear model.

As discussed elsewhere herein methods described herein a number of types of answers are suitable for use in the methods, e.g., answer described herein, e.g., an answer which is the product of an operation on the level of expression of a plurality of genes.

As discussed elsewhere herein, the types of answers and/or observations can be the level of expression of a gene or genes described herein.

In another aspect, the invention features, a method of selecting a glycoprotein for manufacture in a cell population. The method comprises:

(a) acquiring a cell population quality attribute profile, comprising a set of answers, wherein an answer is expressed in terms of a glycostructure and is the product of an operation on a plurality of observations, for a cell population;

(b) acquiring the identities of a plurality of glycostructures;

(c) comparing the acquired profile with the identities acquired in (b);

(d) responsive to the comparison (e.g., when the identities acquired in (b) and the acquired profile show a preselected relationship with one another, e.g., the former includes the latter), selecting one of the plurality glycostructures for production in said cell population; and

(e) making a glycoprotein having the selected glycostructure in said cell population.

As discussed elsewhere herein a method can require one or more “acquiring” steps, e.g., acquiring a profile or acquiring the identity of a selected post-translational modification. In an embodiment acquiring a value comprises subjecting a sample to a process which results in a physical change in the sample or another substance, e.g., an analytical reagent or a device used in the analysis, e.g., such an analysis described herein. In other embodiments, e.g., in embodiments where the method includes the production of a glycoprotein, or culturing a cell, harvesting a glycoprotein or purifying a glycoprotein, or another step which results in a transformation of an entity used in the method, e.g., a cell, glycoprotein or reagent, the acquiring step may be a step that can be yielded without such a transformation, e.g., by inspection, comparing or receiving information from another party.

As discussed elsewhere herein, an observation can be expressed in terms of glycan structure, e.g., a glycan structure disclosed herein.

As discussed elsewhere herein, an observation can be expressed in terms other than glycan structure, e.g., the level of gene expression, e.g., a gene discussed herein, or a metabolite, e.g., a metabolite discussed herein.

As discussed elsewhere herein methods described herein can be used with a range of cell populations, e.g., a CHO or other cell population described herein.

As discussed elsewhere herein methods described herein a number of types of operation are suitable for use in the methods, e.g., operations discussed herein, e.g., an arithmetic combination or linear model.

As discussed elsewhere herein methods described herein a number of types of answers are suitable for use in the methods, e.g., answer described herein, e.g., an answer which is the product of an operation on the level of expression of a plurality of genes.

As discussed elsewhere herein, the types of answers and/or observations can be the level of expression of a gene or genes described herein.

In one aspect, the disclosure features a data base comprising a plurality of records for isolates of a cell population of a preselected cell population, e.g., CHO cells, wherein each record comprises an identifier for a unique (as opposed to others in the plurality) isolate of said preselected cell type and an identifier for a cell population quality attribute profile unique for the isolate, and wherein said cell population quality attribute profile for each entry is unique (as opposed to others in the plurality) for the isolate.

In an embodiment, preselected cell type is CHO or other cell population described herein.

A data base comprising a plurality of records, each record of the plurality corresponding to an isolate of a cell population of a preselected cell population, e.g., CHO cells, wherein said plurality of records comprises:

a first record comprising an identifier for a first isolate of said preselected cell type and an identifier for a first cell population quality attribute profile for said first isolate,

a second record comprising an identifier for a second isolate of said preselected cell type and an identifier for a second cell population quality attribute profile unique for second isolate,

wherein the cell population quality attribute profile in each of the records of said plurality of records is distinct for each isolate in the plurality is different from the cell population quality attribute profile for each other isolate in the plurality.

In an embodiment, the data base comprises records for at least 5, 10, or 20 isolates.

In one aspect, the invention features a method of making a glycoprotein having a selected glycan component and/or glycan complement, or providing or selecting a CHO cell population from a plurality of CHO populations, e.g., for use in making a glycoprotein having a selected glycan component and/or glycan complement. The method comprises:

(a) acquiring, directly or indirectly, the identity of a CHO cell population for production of said glycoprotein, wherein the identity is acquired or determined by a method described herein, e.g., by
(i) acquiring, for each of a plurality of isolates or aliquots of a first CHO cell population, e.g., a CHO cell population described herein, a value which is expressed in terms of glycan component and/or glycan complement, which value is a function of a plurality of distinct observations that include the level of expression of a plurality of genes and the level of expression of a plurality of different glycostructures, glycan structures, glycan components, glycan complement or combinations thereof, to provide a set of values for said first CHO cell population;
(ii) acquiring, for each of a plurality of isolates or aliquots of a second CHO cell population, e.g., a CHO cell population described herein, a value which is expressed in terms of glycan component and/or glycan complement, which value is a function of a plurality of distinct observations that include the level of expression of a plurality of genes and/or metabolites and the level of expression of a plurality of different glycostructures, glycan structures, glycan components, glycan complement or combinations thereof, to provide a set of values for said second CHO cell population, wherein said second CHO cell population differs from said first CHO cell population, e.g., by a naturally acquired or intentionally induced mutation;
(iii) comparing a value for a selected glycan component or glycan complement with the set of values for said first CHO cell population and with the set of values for the second CHO cell population;
(iv) responsive to said comparison, selecting said first or second CHO cell population.

In one embodiment, the selected glycan component and/or glycan complement is a glycan component or glycan complement of a biologic therapeutic glycoprotein, e.g., a marketed biologic therapeutic glycoprotein, and the CHO cell population selected has a set of values that indicates that produces a glycoprotein having the glycan component and/or glycan complement of the marketed biologic therapeutic glycoprotein.

In an embodiment, the glycoprotein is a therapeutic antibody, Fc-receptor fusion, hormone, cytokine. In one embodiment, the glycoprotein is a biosimilar or biogeneric version of a marketed therapeutic biologic product.

In one embodiment, the method is a method of providing or selecting a CHO cell population, e.g., for use in making a glycoprotein having a selected post-translational modification (e.g., a selected glycostructure, glycan complement, glycan component, e.g., with a selected glycan structure) and the method further comprises (b) culturing said selected CHO cell population.

In an embodiment, the method is a method of making a glycoprotein having a selected glycan complement and/or glycan component and, and the method further comprises (b) making a glycoprotein having a selected glycan complement and/or glycan component in said selected CHO cell population.

In one embodiment, the method can further comprise genetically modifying the identified CHO cell population to express said glycoprotein, e.g., introducing a nucleic acid that encodes all or part of said glycoprotein into said identified CHO cell population prior to step (b).

In an embodiment, one of the CHO cell populations of the plurality of CHO cell populations is a CHO K1 cell line.

In an embodiment, one of the CHO cell populations of the plurality of CHO cell populations is a CHO S cell line.

In an embodiment, one of the CHO cell populations of the plurality of CHO cell populations is a DG44 cell line.

In an embodiment, one of the CHO cell populations of the plurality of CHO cell populations is a DHFR(−) cell line.

In an embodiment, one of the CHO cell populations of the plurality of CHO cell populations is a CHO GS cell line.

In an embodiment, a set of values is acquired for a plurality, e.g., at least 3, 4, 5, 6, 7, 8, 9, 10 of CHO cell populations. In an embodiment, a set of values is acquired for a plurality of CHO cell populations including a CHO K1 cell line, a CHO S cell line, a DG44 cell line and a DHFR(−) cell line.

As discussed elsewhere herein, an observation can be expressed in terms of glycan structure, e.g., a glycan structure disclosed herein.

As discussed elsewhere herein, an observation can be expressed in terms of the level of gene expression, e.g., a gene discussed herein, or a metabolite, e.g., a metabolite discussed herein.

In one embodiment, the observations for each CHO cell population include at least 2, 3, 4, 5, 6, 7, 8, 9, 10 or more observations of the expression levels of genes. In an embodiment, the observations for each cell population include at least 2, 3, 4, 5, 6, 7, 8, 9, 10 or more observations regarding the levels of a glycan metabolite.

In an embodiment, an observation is the level of 4,4,1,0,0.

In an embodiment, an observation is the level of 4,4,1,1,0.

In an embodiment, an observation is the level of 4,5,1,0,0.

In an embodiment, an observation is the level of 4,5,1,1,0.

In an embodiment, an observation is the level of 4,5,1,2,0.

In an embodiment, an observation is the level of 5,5,1,0,0.

In an embodiment, an observation is the level of 5,6,1,0,0.

In an embodiment, an observation is the level of 5,6,1,1,0.

In an embodiment, an observation is the level of 5,6,1,2,0.

In an embodiment, an observation is the level of 5,6,1,3,0.

In an embodiment, an observation is the level of 6,6,1,1,0.

In an embodiment, an observation is the level of 6,6,1,2,0.

In an embodiment, an observation is the level of 6,7,1,1,0.

In an embodiment, an observation is the level of 6,7,1,2,0.

In an embodiment, an observation is the level of 6,7,1,3,0.

In an embodiment, an observation is the level of 6,7,1,4,0.

In an embodiment, an observation is the level of expression of a glycosyltransferase.

In an embodiment, an observation is the level of expression of a gene involved in glycan biosynthesis.

In an embodiment, an observation is the level of a metabolite.

In an embodiment, an observation is the level of UMP.

In an embodiment, an observation is the level of GTP.

In an embodiment, an observation is the level of UDP-Gal.

In an embodiment, an observation is the level of GDP-Fuc.

In one aspect, the invention features, a method of making a glycoprotein having a selected glycan complement and/or glycan component, or providing or selecting a CHO cell population, e.g., for use in making a glycoprotein having a selected glycan complement and/or glycan component. The method comprises:
(i) acquiring a cell population quality attribute profile (a profile), comprising a set of answers, wherein an answer is expressed in terms of a post-translational modification and is the product of an operation on a plurality of observations, for each of a plurality of CHO cell populations, said acquired profiles forming a plurality of distinct profiles;
(ii) acquiring the identity of glycan complement and/or glycan component;
(iii) comparing the acquired profile with the identity acquired in (ii);
(iv) responsive to the comparison (e.g., when the acquired profile and the identity acquired of the selected glycan component and/or glycan complement show a preselected relationship with one another, e.g., the former includes the latter), selecting one of the plurality of CHO cell populations for production of the subject glycoprotein; selecting or providing the CHO cell population to make said glycoprotein and/or making said glycoprotein having the selected glycan component and/or glycan complement in said selected CHO cell population. In some embodiments, the method further comprises introducing a nucleic acid that encodes all or part of said glycoprotein into said selected CHO cell population.

In one embodiment, the selected glycan component and/or glycan complement is a glycan component or glycan complement of a biologic therapeutic glycoprotein, e.g., a marketed biologic therapeutic glycoprotein, and the CHO cell population selected has a set of values that indicates that produces a glycoprotein having the glycan component and/or glycan complement of the marketed biologic therapeutic glycoprotein.

In an embodiment, the glycoprotein is a therapeutic antibody, Fc-receptor fusion, hormone, cytokine. In one embodiment, the glycoprotein is a biosimilar or biogeneric version of a marketed therapeutic biologic product.

In one embodiment, the method is a method of providing or selecting a CHO cell population, e.g., for use in making a glycoprotein having a selected glycan complement and/or glycan component and the method further comprises culturing said selected CHO cell population.

In an embodiment, the method is a method of making a glycoprotein having a selected glycan complement and/or glycan component and, and the method further comprises making a glycoprotein having a selected glycan complement and/or glycan component in said selected CHO cell population.

In one embodiment, the method can further comprise genetically modifying the selected CHO cell population to express said glycoprotein, e.g., introducing a nucleic acid that encodes all or part of said glycoprotein into said identified CHO cell population.

In an embodiment, one of the CHO cell populations of the plurality of CHO cell populations is a CHO K1 cell line.

In an embodiment, one of the CHO cell populations of the plurality of CHO cell populations is a CHO S cell line.

In an embodiment, one of the CHO cell populations of the plurality of CHO cell populations is a DG44 cell line.

In an embodiment, one of the CHO cell populations of the plurality of CHO cell populations is a DHFR(−) cell line.

In an embodiment, one of the CHO cell populations of the plurality of CHO cell populations is a CHO GS cell line.

In an embodiment, a set of answers is acquired for a plurality, e.g., at least 3, 4, 5, 6, 7, 8, 9, 10 of CHO cell populations. In an embodiment, a set of answers is acquired for a plurality of CHO cell populations including a CHO K1 cell line, a CHO S cell line, a DG44 cell line and a DHFR(−) cell line.

As discussed elsewhere herein, an observation can be expressed in terms of glycan structure, e.g., a glycan structure disclosed herein.

As discussed elsewhere herein, an observation can be expressed in terms of the level of gene expression, e.g., a gene discussed herein, or a metabolite, e.g., a metabolite discussed herein.

In one embodiment, the observations for each CHO cell population include at least 2, 3, 4, 5, 6, 7, 8, 9, 10 or more observations of the expression levels of genes. In an embodiment, the observations for each CHO cell population include at least 2, 3, 4, 5, 6, 7, 8, 9, 10 or more observations regarding the levels of a glycan metabolite.

In an embodiment, an observation is the level of 4,4,1,0,0.

In an embodiment, an observation is the level of 4,4,1,1,0.

In an embodiment, an observation is the level of 4,5,1,0,0.

In an embodiment, an observation is the level of 4,5,1,1,0.

In an embodiment, an observation is the level of 4,5,1,2,0.

In an embodiment, an observation is the level of 5,5,1,0,0.

In an embodiment, an observation is the level of 5,6,1,0,0.

In an embodiment, an observation is the level of 5,6,1,1,0.

In an embodiment, an observation is the level of 5,6,1,2,0.

In an embodiment, an observation is the level of 5,6,1,3,0.

In an embodiment, an observation is the level of 6,6,1,1,0.

In an embodiment, an observation is the level of 6,6,1,2,0.

In an embodiment, an observation is the level of 6,7,1,1,0.

In an embodiment, an observation is the level of 6,7,1,2,0.

In an embodiment, an observation is the level of 6,7,1,3,0.

In an embodiment, an observation is the level of 6,7,1,4,0.

In an embodiment, an observation is the level of expression of a glycosyltransferase.

In an embodiment, an observation is the level of expression of a gene involved in glycan biosynthesis.

In an embodiment, an observation is the level of a metabolite.

In an embodiment, an observation is the level of UMP.

In an embodiment, an observation is the level of GTP.

In an embodiment, an observation is the level of UDP-Gal.

In an embodiment, an observation is the level of GDP-Fuc.

As discussed elsewhere herein, a number of types of observations are suitable for use in the methods, e.g., operations discussed herein, e.g., an arithmetic combination or linear model.

As discussed elsewhere herein, a number of types of answers are suitable for use in the methods, e.g., an answer described herein, e.g., an answer which is the product of an operation on the level of expression of a plurality of genes.

As discussed elsewhere herein, the types of answers and/or observations can be the level of expression of a gene or genes described herein.

Headings and identifiers, e.g., (a), (b), (i) etc, are presented merely for ease of reading the specification and claims. The use of headings or identifiers in the specification or claims does not require the steps or elements be performed in alphabetical or numerical order or the order in which they are presented.

Other features and advantages of the invention will be apparent from the following detailed description, and from the claims.

### DESCRIPTION

The drawings are first briefly described:

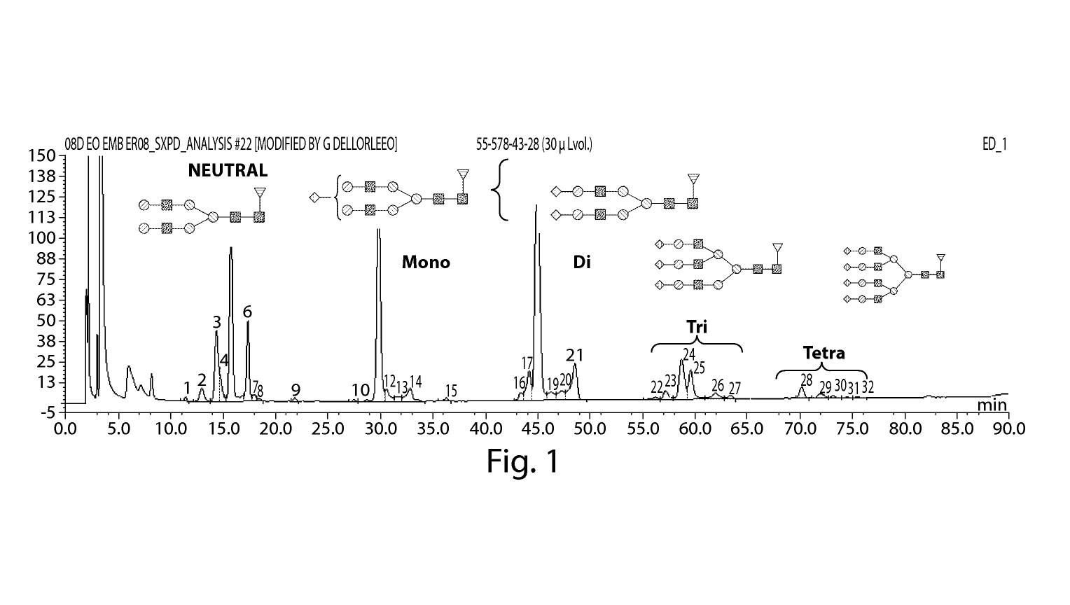

FIG. 1 is an illustrative chromatogram of glycans from the isolated glycoprotein which were released, labeled and analyzed by LC and LC/MS;

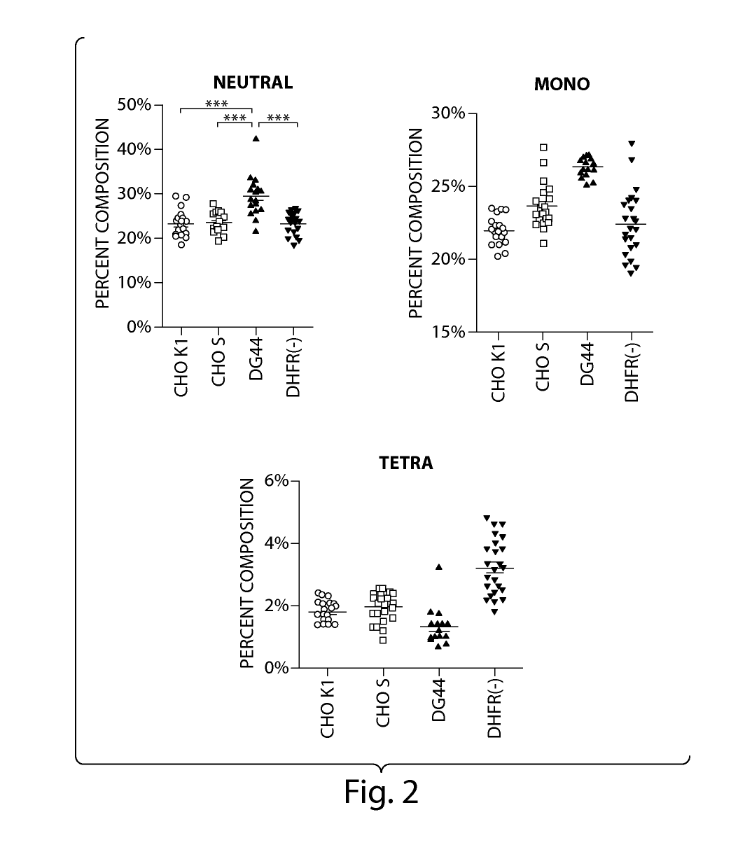

FIG. 2 is a depiction of illustrative LC data of the distribution of the product from CHO clones;

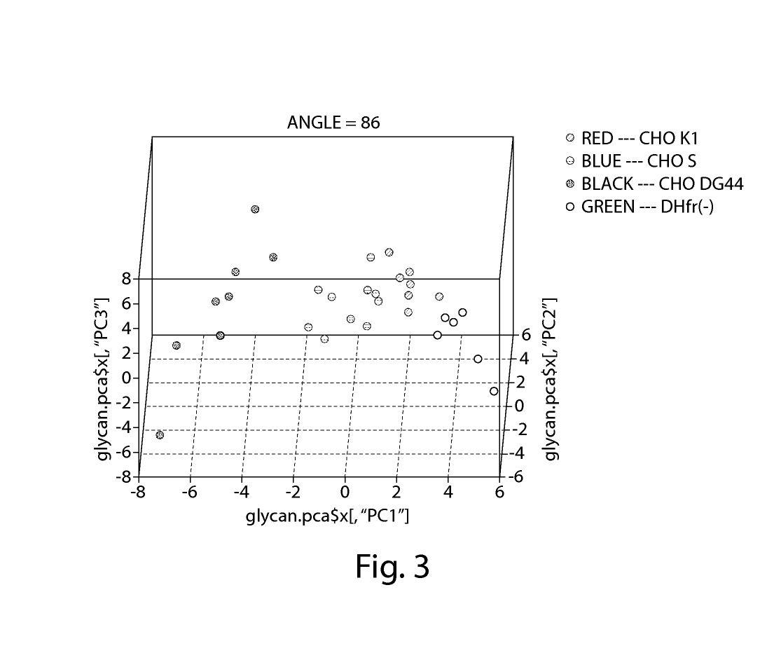

FIG. 3 is a plot of PCA analysis for the cell population quality attribute profiles (CPQAP) for each of the cell types, CHO K1, CHO S, CHO DG44 and DHfr(−).

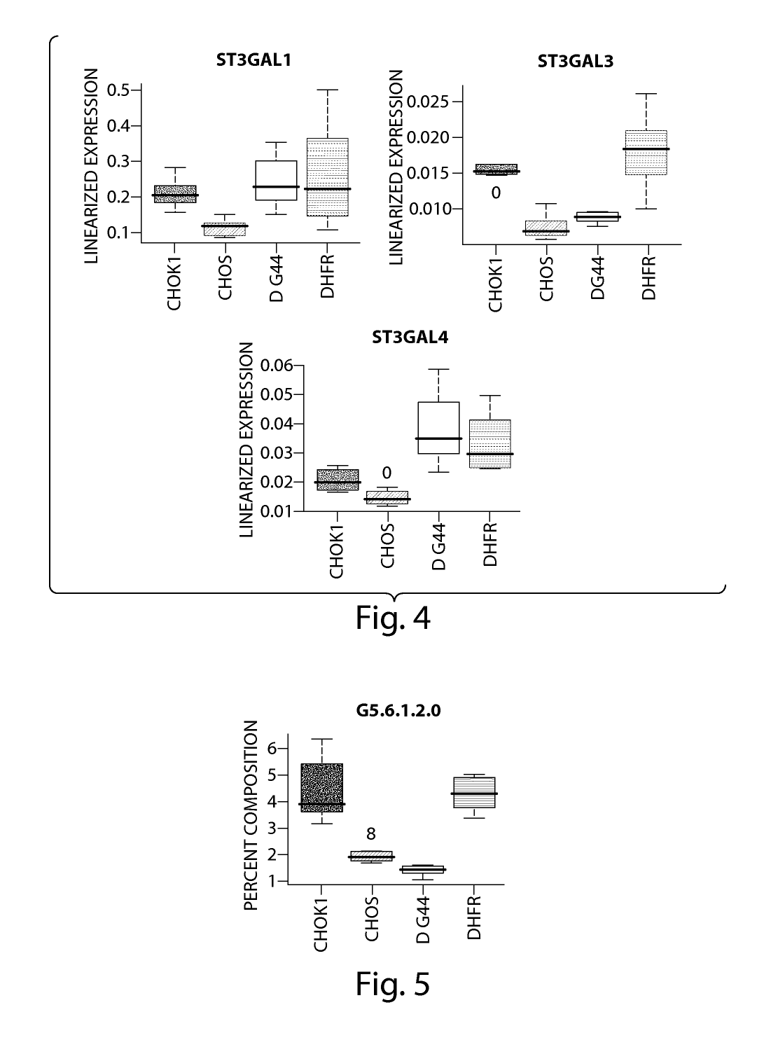

FIG. 4 is a depiction of expression levels.

FIG. 5 is a depiction of expression levels.

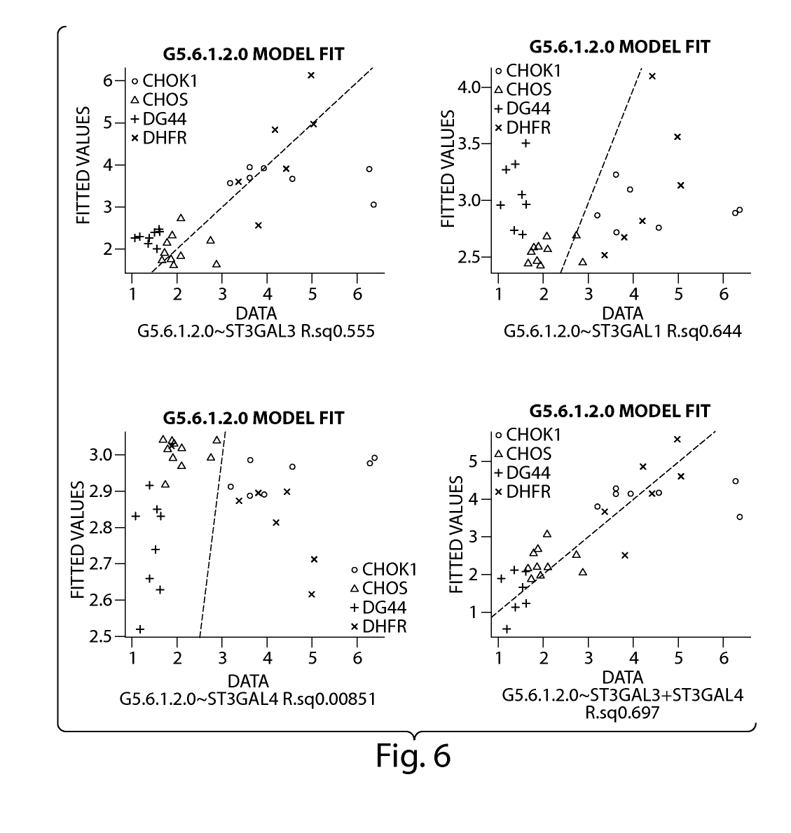

FIG. 6 is a linear model utilizing ST3GAL3 expression to compute the level of glycan 5,6,1,2,0 produced.

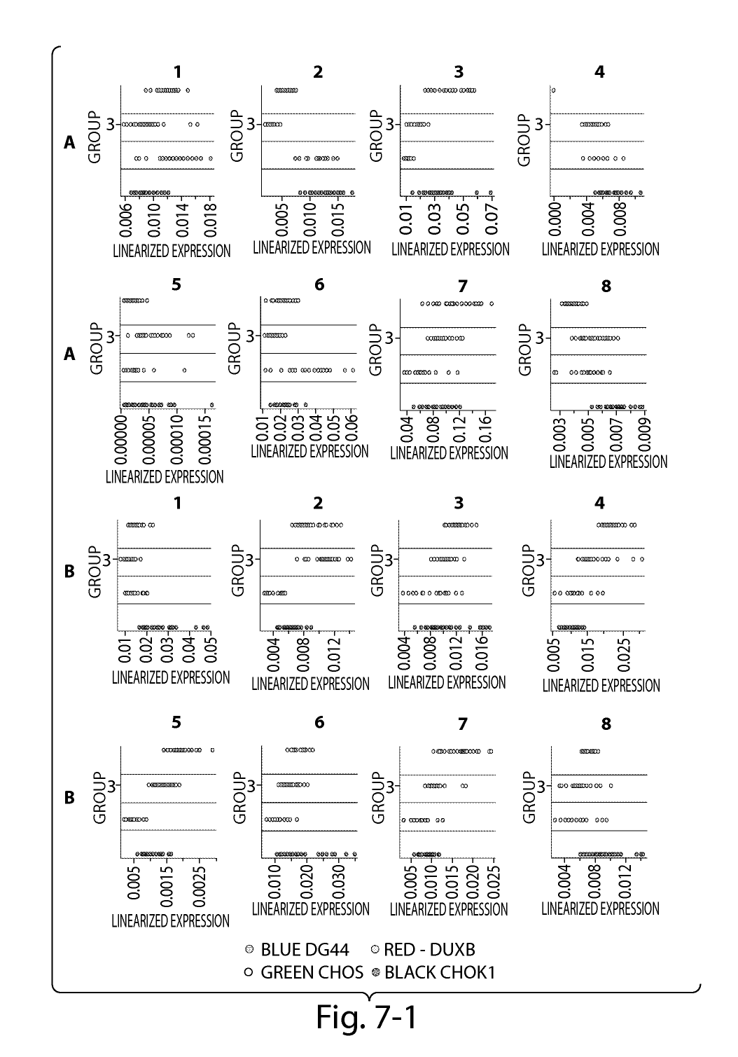

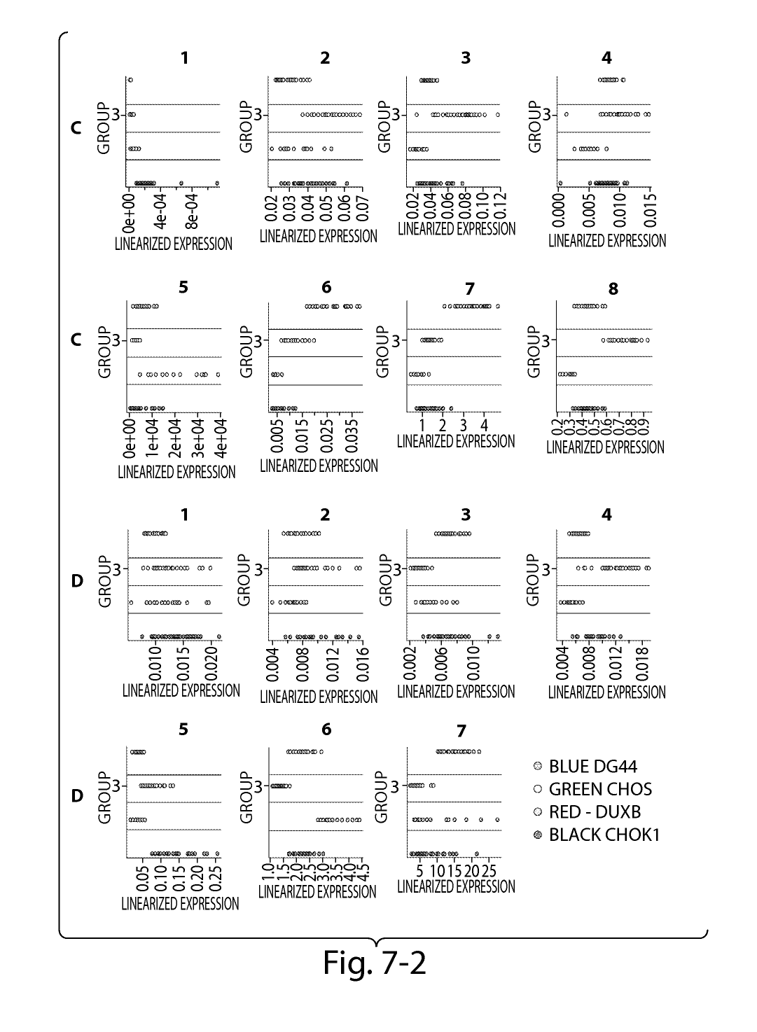

FIG. 7 is a depiction of the distribution of transcripts related to glycosylation across the clones (each dot) from each cell line background clustered for each transcript.

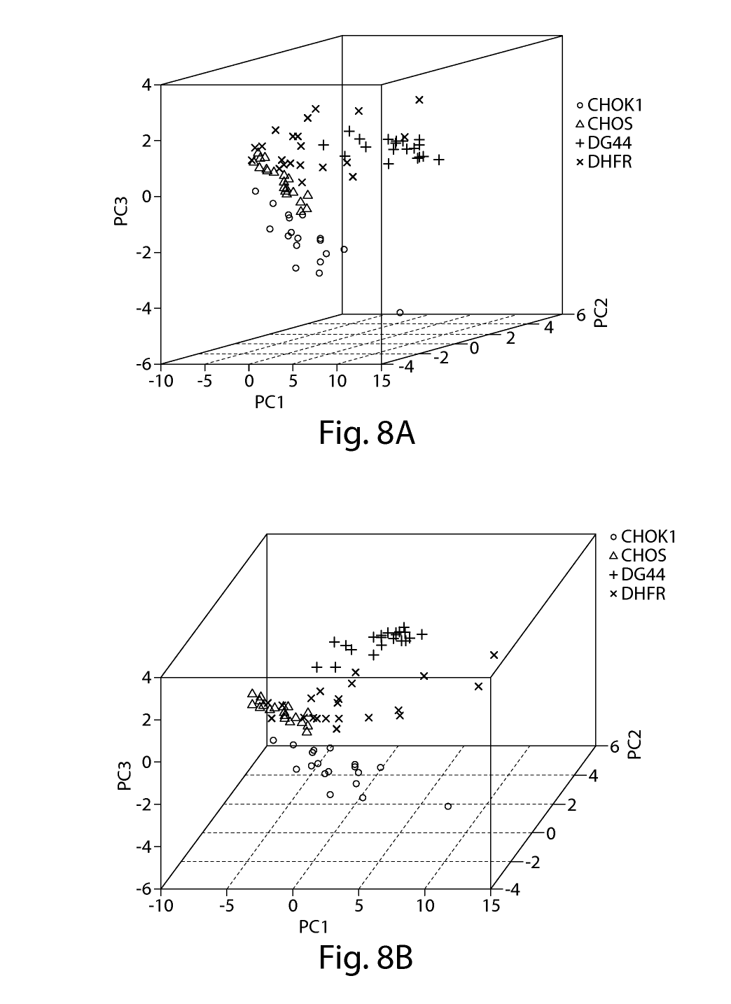

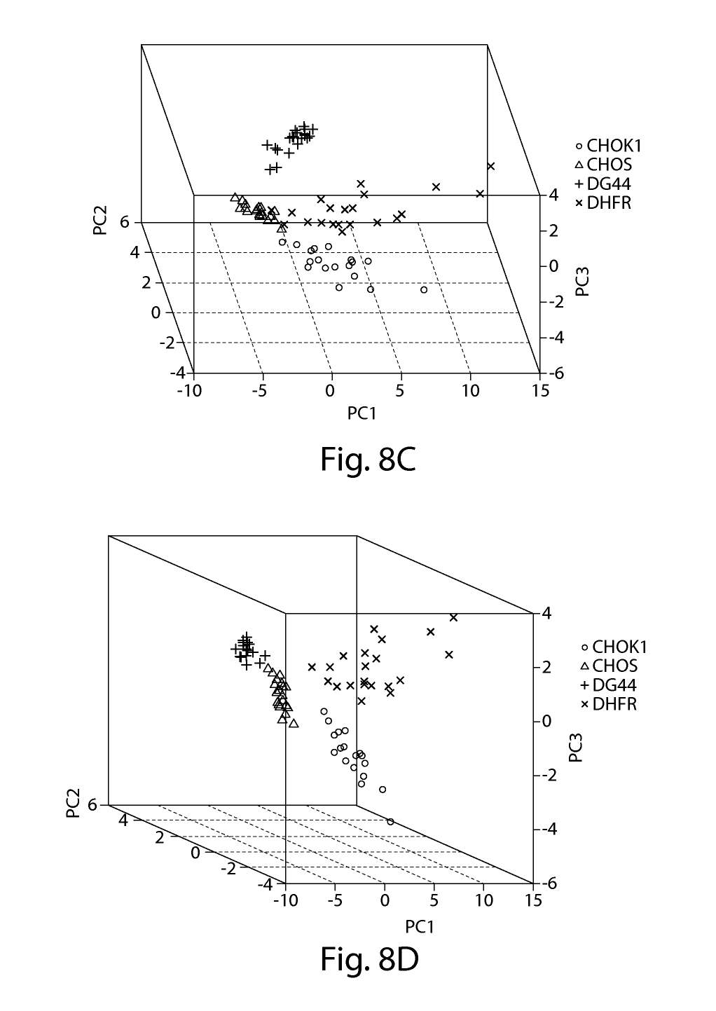

FIG. 8 is a depiction of PCA analysis of transcripts of glycorelated genes derived from each of the clones form the CHO cell line backgrounds, circles CHOK1, triangles CHOS, plus DG44

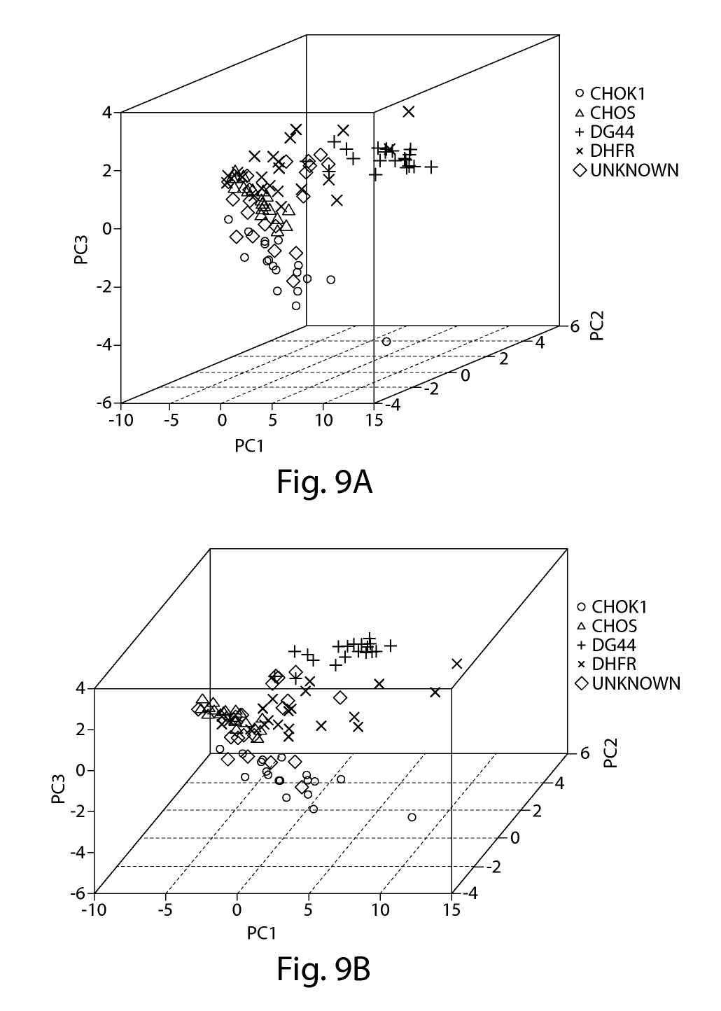

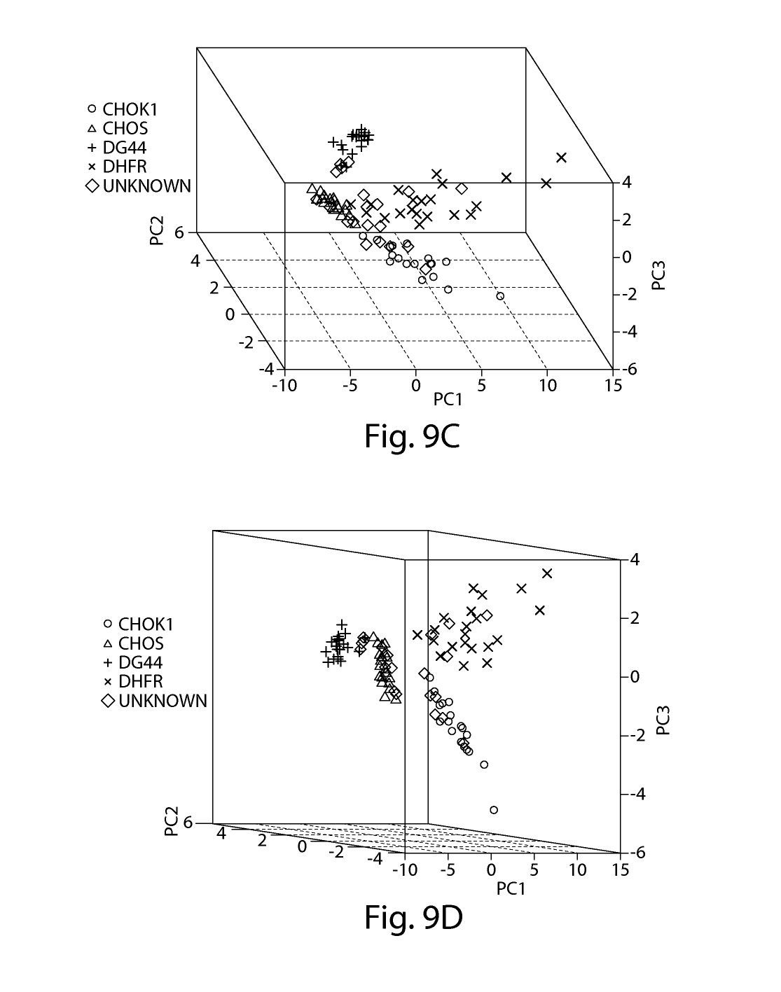

FIG. 9 is a depiction of the unknowns superimposed on the cell population quality attribute profiles for each of the four cell types.

### DEFINITIONS

As used herein, “acquiring a value” refers to any process that results in possession of the value. In an embodiment the value is “directly acquired” by performing one or more physically transforming steps, e.g., on a sample, e.g., a glycoprotein sample, a cell extract, or a sample of cells, e.g., a cell line. The process thus results in a physical change in the sample or another substance, e.g., an analytical reagent or a device used in the process. Such methods, by way of example, comprise: analytical methods; preparatory methods; and manipulations of cells, e.g., extraction or purification of components, e.g., nucleic acid, e.g., mRNA or DNA, or protein, from a cell, or culturing cells. In these methods typically include one or more of the following: separating a substance, e.g., an analyte, or a fragment or other derivative thereof, from another substance, combining a substance, e.g., an analyte, or fragment or other derivative thereof, with another substance, e.g., a buffer, solvent, or reactant; or changing the structure of an analyte, or a fragment of other derivative thereof, e.g., by breaking or forming a covalent or non covalent bond, between a first and a second atom of the substance, e.g., an analyte. The value can also be “indirectly acquired.” Indirect acquisition comprises receiving the value, e.g., from another party, e.g., a party that directly acquired the value. Typically, even in embodiments characterized by indirect acquisition, some party has subjected a sample to a process as described above, which results in a physical change in the sample or another substance. In an embodiment a party that practices the method of evaluating instructs another party to perform the process, and e.g., a party that practices the method receives the value. In an embodiment a value can be an expression of whether or to what degree a cell or cell line possesses a characteristic, e.g., a glycan structure related characteristic, e.g., the a level of a transcript, the ability to make a glycoprotein having a preselected glycan structure, a preselected level of a glycan structure, a preselected ratio of a first to a second glycan structure, or a preselected glycan structure at a preselected location.

A “cell population quality attribute profile” (CPQAP) comprises a set of answers for a cell population. A set comprises at least two answers. Typically a set comprises an answer for a first cell, e.g., a first isolate or aliquot of a cell population, and an answer for a second cell, e.g., a second isolate or aliquot of the cell population. An answer, which is expressed in terms of a post-translational modification, e.g., a glycan structure, is the product of operation on a plurality of observations (e.g., measurements or determined characteristics). An operation relates the observations to a post-translational modification, e.g., a glycan structure. In an embodiment the observations are expressed in terms of a post-translational modification, e.g., glycan structure. In an embodiment the observation is not expressed in terms of a post-translational modification, e.g., glycan structure, e.g., they are expressed in terms of gene expression, and the operation also converts them to units of a post-translational modification, e.g., glycan structure. Exemplary operations include correlation of observation(s) to a post-translational modification, e.g., glycan structure, e.g., by use of a look-up table or equivalent tool; use of the observations as inputs into a model, e.g., a linear model, which relates observations to post-translational modification, e.g., a glycan structure; or, e.g., when the observations are themselves expressed in terms of a post-translational modification, e.g., glycan structure, combination, e.g., by addition, of observations. The observation can be obtained by principle component analysis. The set of answers comprising a cell population quality attribute profile, if viewed as continuous, can be visualized/analyzed as defining a discrete space occupied by the cell population. E.g., the set of answers can be depicted in n dimensions and occupy a space of n dimensions, e.g., if depicted in 3 dimensions the set defines a 3 dimensional space.

In a “plurality of distinct cell population quality attribute profiles” as that term is used herein, each cell population quality attribute profile in the plurality is distinct from each other CPQAP in the plurality, e.g., at least one answer of a first profile differs from at least one answer of a second profile.

In an embodiment an answer is a direct indication of the state of a post-translational modification, e.g., a glycostructure, e.g., the presence or level of a glycostructure, a cell having level x of glycan x and level y of glycan y. A selected post-translational modification, e.g., a glycostructure, e.g., a glycostructure present on a reference protein, is a post-translational modification, e.g., a glycostructure, which is to be included on a protein. If the set of answers includes the selected post-translational modification, e.g., a glycostructure, or to put it another way, if the selected glycostructure falls within the cell population quality attribute profile, then the cell population can be selected for production of a glycoprotein having the selected post-translational modification, e.g., a glycostructure. Comparison of the post-translational modification, e.g., a glycostructure, with a plurality of cell population quality attribute profiles allows for selection of a cell population to optimize production of a protein having the selected post-translational modification, e.g., a glycostructure.

A “distinct isolate” as used herein, refers to relationship between a first cell or group of cells and a second cell or group of cells. Distinct isolates have a common cellular ancestor but where the founder cells of each distinct isolate are separated by at least 1, 10, 20, 50, 100, 500, 1,000, 5,000 or 10,000 cycles of cell divisions. To illustrate, a parental cell divides to give two F1 cells, each F1 cell divides to give two F2 cells, each F2 cell divides to give two F3 cells. There are three cycles of cell division between the parental cell and the F3 cell. Typically, the common cellular ancestor is a cell, e.g., a cultured cell, that has been removed from a multicellular organism, e.g., an insect or animal, e.g., a mammal or primate, excluding as common cellular ancestors, precursor cells of the animal or ancestors of the animal from which the common cellular ancestor is taken.

An “observation,” as used herein, is a value for a parameter, e.g., a measurement, determined or observed value for a parameter, related to a property of a cell.

“Closely related cell populations” as used herein, refer to cell populations that have one or more, and in embodiments two or more, or all, of the following properties: they are from the same species; they are from the same tissue type; they are of the same cell type, e.g., they are stromal cells; they have the same transformation state (e.g., are both transformed and show essentially immortal growth in culture or both are incapable of immortalized growth, or both have growth rates that are within 2× of each other on a selected medium). In embodiments their founder cells were separated from one another by less than 1,000, and in embodiments less than 500, or 100 cycles of cell division.

A “glycostructure”, as used herein, refers to one or more elements of the glycan complement of a glycoprotein or to a selected glycan structure. It can, e.g., refer to a single monosaccharide, a single glycan component (e.g., the presence of high mannose structures), or to the entire glycan complement of a glycoprotein, or two a particular glycan structure, e.g., a high mannose glycan component.

“Glycan complement” as used herein refers to all of the glycan components of a glycoprotein. In the case of a protein having a single glycosylation site, the glycan component attached thereto forms the glycan complement. In the case of a protein having more than one glycosylation site, the glycan complement is made up of the glycan components attached at all of the sites. A “component of the glycan complement” refers to a subset of the glycan components making up the glycan complement, e.g., one or more glycan components attached to its or their respective glycosylation site or sites. The glycan complement can be the average of all of the glycan components of all of the glycoproteins in the mixture. The glycan complement can also be all of the glycan components associated with a glycoform within a glycoprotein mixture.

“Glycan component” as used herein, refers to a sugar moiety, e.g., a monosaccharide, oligosaccharide or polysaccharide (e.g., a disaccharide, trisaccharide, tetrasaccharide, etc.) attached to a protein at one site. In embodiments the attachment is covalent and the glycan component is N- or O-linked to the protein. Glycan components can be chains of monosaccharides attached to one another via glycosidic linkages. Glycan components can be linear or branched.

“Glycan structure” as used herein refers to the structure of a glycan complement, component of a glycan complement, or glycan component. Elements of glycan structure include one or more of the following:

the presence, absence, or level of glycosylation at one or more sites, e.g., one or more sites for N-linked or O-linked glycosylation;

N- or O-linkage;

length (number of monosaccharide moieties);

placement or position of a monosaccharide, e.g., a galactosyl moiety, within a chain;

saccharide content (e.g., the amounts or ratios of the monosaccharide components in a particular glycan);

saccharide sequence (e.g., the order of monosaccharide subunits in a glycan moiety);

the presence, absence or amount of a terminal or penultimate saccharide subunit;

the number, placement, and type (e.g., the presence, absence or amount of bisecting GlcNAc or mannose structures) of branch points;

the presence, absence or level of a complex structure, e.g., biantennary structure, triantennary structure, tetraantennary structure, etc;

the presence, absence or level of a high mannose or a hybrid structure;

the relationship between monosaccharide moieties (e.g., linkages between monosaccharide moieties, isomers and branch points);

the presence, absence, position, or number of a selected monosaccharide, e.g., a galactosyl moiety, fucosyl moiety, GlcNAc moiety, or mannosyl moiety;

the presence, absence, position or number of a selected structure, e.g., a mono-galactosylated, digalactosylated, or polygalactosylated structure. Other nonlimiting examples include any other structure found on naturally occurring glycoproteins; and

heterogeneity or homogeneity across one or more sites (e.g., diversity across the entire protein, e.g., the degree of occupancy of potential glycosylation sites of a protein (e.g., the degree of occupancy of the same potential glycosylation site between two or more of the particular protein backbones in a plurality of molecules and the degree of occupancy of one potential glycosylation site on a protein backbone relative to a different potential glycosylation site on the same protein backbone).

A glycan structure can be described in terms of a comparison of the presence, absence or amount of a first glycan structure to a second glycan structure. For example, the presence, absence or amount of sialic acid relative to the presence, absence or amount of fucose. In other examples, the presence, absence or amount of a sialic acid such as N-acetylneuraminic acid can be compared, e.g., to the presence, absence or amount of a sialic acid derivative such as N-glycolylneuraminic acid.

Glycan structures can be described, identified or assayed in a number of ways. A glycan structure can be described, e.g., in defined structural terms, e.g., by chemical name, or by a functional or physical property, e.g., by molecular weight or by a parameter related to purification or separation, e.g., retention time of a peak in a column or other separation device. In embodiments a glycan structure can, by way of example, be a peak or other fraction (representing one or more species) from glycan structures derived from a glycoprotein, e.g., from an enzymatic digest.

“Monosaccharide” as used herein refers to the basic unit of a glycan component and in embodiments, a moiety that is transferred by a glycosyltransferase onto a substrate. Monosaccharides, as used herein, include naturally and non-naturally occurring monosaccharides. Exemplary monosaccharide moieties include glucose (Glc), N-acetylglucosamine (GlcNAc), mannose (Man), N-acetylmannosamine (ManNAc), galactose (Gal), N-acetylgalactosamine (GalNAc), fucose (Fuc), sialic acid (NeuAc) and ribose, as well as derivatives and analogs thereof. Derivatives of various monosaccharides are known. For example, sialic acid encompasses over thirty derivatives with N-acetylneuraminic acid and N-glycolylneuraminic acid forming the core structures. Examples of sialic acid analogs include those that functionally mimic sialic acid, but are not recognized by endogenous host cell sialylases. Other examples of monosaccharide analogs include, but are not limited to, N-levulinoylmannosamine (ManLev), Neu5Acα-methyl glycoside, Neu5Acβ-methyl glycoside, Neu5Acα-benzyl glycoside, Neu5Acβ-benzyl glycoside, Neu5Acα-methylglycoside methyl ester, Neu5Acα-methyl ester, 9-O-Acetyl-N-acetylneuraminic acid, 9-O-Lactyl-N-acetylneuraminic acid, N-azidoacetylmannosamine and O-acetylated variations thereof, and Neu5Acα-ethyl thioglycoside.

“High Mannose” as used herein refers to one or a multiple of N-glycan structures including HM3, HM4, HM5, HM6, HM7, HM8, and HM9 containing 3, 4, 5, 6, 7, 8, or 9 mannose residues respectively.

### Cells & Cell Lines

Methods described herein use cells to produce glycoproteins having selected post-translational modifications (e.g., glycostructures). Examples of cells and cell lines useful in these and other methods described herein follow.

The cell useful in the methods described herein can be eukaryotic or prokaryotic, as long as the cell provides or has added to it the appropriate enzymes to activate and attach (or remove) saccharides present in the cell or saccharides present in the cell culture medium or fed to the cells. Examples of eukaryotic cells include yeast, insect, fungi, plant and animal cells, especially mammalian cells. Suitable mammalian cells include any normal mortal or normal or abnormal immortal animal or human cell, including: monkey kidney CV1 line transformed by SV40 (COS-7, ATCC CRL 1651); human embryonic kidney line (293) (Graham et al., J. Gen. Virol. 36:59 (1977)); baby hamster kidney cells (BHK, ATCC CCL 10); Chinese Hamster Ovary (CHO), e.g., DG44, DUKX-V11, GS-CHO (ATCC CCL 61, CRL 9096, CRL 1793 and CRL 9618); mouse sertoli cells (TM4, Mather, Biol. Reprod. 23:243 251 (1980)); monkey kidney cells (CV1 ATCC CCL 70); African green monkey kidney cells (VERO-76, ATCC CRL 1587); human cervical carcinoma cells (HeLa, ATCC CCL 2); buffalo rat liver cells (BRL 3A, ATCC CRL 1442); human lung cells (W138, ATCC CCL 75); human liver cells (Hep G2, HB 8065); mouse melanoma cells (NSO); mouse mammary tumor (MMT 060562, ATCC CCL51), TR1 cells (Mather, et al., Annals N.Y. Acad. Sci. 383:44 46 (1982)); canine kidney cells (MDCK) (ATCC CCL 34 and CRL 6253), HEK 293 (ATCC CRL 1573), WI-38 cells (ATCC CCL 75) (ATCC: American Type Culture Collection, Rockville, Md.), MCF-7 cells, MDA-MB-438 cells, U87 cells, A127 cells, HL60 cells, A549 cells, SP10 cells, DOX cells, SHSY5Y cells, Jurkat cells, BCP-1 cells, GH3 cells, 9L cells, MC3T3 cells, C3H-10T1/2 cells, NIH-3T3 cells, C6/36 cells, human lymphoblast cell lines (e.g. GEX) and PER.C6® cells. The use of mammalian tissue cell culture to express polypeptides is discussed generally in Winnacker, FROM GENES TO CLONES (VCH Publishers, N.Y., N.Y., 1987).

Exemplary plant cells include, for example, Arabidopsis thaliana, rape seed, corn, wheat, rice, tobacco etc.) (Staub, et al. 2000 Nature Biotechnology 1(3): 333-338 and McGarvey, P. B., et al. 1995 Bio-Technology 13(13): 1484-1487; Bardor, M., et al. 1999 Trends in Plant Science 4(9): 376-380). Exemplary insect cells (for example, Spodoptera frugiperda Sf9, Sf21, Trichoplusia ni, etc. Exemplary bacteria cells include Escherichia coli. Various yeasts and fungi such as Pichia pastoris, Pichia methanolica, Hansenula polymorpha, and Saccharomyces cerevisiae can also be selected.

Culture Media and Processing

The methods described herein can include determining and/or selecting media components or culture conditions which result in the production of a desired glycostructure. Culture parameters that can be determined include media components, pH, feeding conditions, osmolarity, carbon dioxide levels, agitation rate, temperature, cell density, seeding density, timing and sparge rate.

Changes in production parameters such the speed of agitation of a cell culture, the temperature at which cells are cultures, the components in the culture medium, the times at which cultures are started and stopped, variation in the timing of nutrient supply can result in variation of a glycan properties of the produced glycoprotein product. Thus, methods described herein can include one or more of: increasing or decreasing the speed at which cells are agitated, increasing or decreasing the temperature at which cells are cultures, adding or removing media components, and altering the times at which cultures are started and/or stopped.

Sequentially selecting a production parameters or a combination thereof, as used herein, means a first parameter (or combination) is selected, and then a second parameter (or combination) is selected, e.g., based on a constraint imposed by the choice of the first production parameter.

Media

The methods described herein can include determining and/or selecting a media component and/or the concentration of a media component that has a positive correlation to a desired glycostruture. A media component can be added in or administered over the course of glycoprotein production or when there is a change in media, depending on culture conditions. Media components include components added directly to culture as well as components that are a byproduct of cell culture.

Media components include, e.g., buffer, amino acid content, vitamin content, salt content, mineral content, serum content, carbon source content, lipid content, nucleic acid content, hormone content, trace element content, ammonia content, co-factor content, indicator content, small molecule content, hydrolysate content and enzyme modulator content.

Physiochemical Parameters

Methods described herein can include selecting culture conditions that are correlated with a desired glycostructure. Such conditions can include temperature, pH, osmolality, shear force or agitation rate, oxidation, spurge rate, growth vessel, tangential flow, DO, CO₂, nitrogen, fed batch, redox, cell density and feed strategy. Examples of physiochemical parameters that can be selected are provided in Table 2.

TABLE 2

| | |
|---|---|
| Temperature | DO |
| pH | CO₂ |
| Osmolality | Nitrogen |
| shear force, or agitation rate | Fed batch |
| Oxidation | Redox |
| Spurge rate | Cell density |
| Growth vessel | Perfusion culture |
| Tangential flow | Feed strategy |
| Batch | |

For example, the production parameter can be culturing a cell under acidic, neutral or basic pH conditions. Temperatures can be selected from 10 to 42° C. For example, a temperature of about 28 to 36° C. does not significantly alter galactosylation, fucosylation, high mannose production, hybrid production or sialylation of glycoproteins produced by a cell (e.g., a CHO cell, e.g., a dhfr deficient CHO cell) cultured at these temperatures. In addition, any method that slows down the growth rate of a cell may also have this effect. Thus, temperatures in this range or methods that slow down growth rate can be selected when it is desirable not to have this parameter of production altering glycosynthesis.

In other embodiments, carbon dioxide levels can be selected which results in a desired glycan characteristic or characteristics. CO₂ levels can be, e.g., about 5%, 6%, 7%, 8%, 9%, 10%, 11%, 13%, 15%, 17%, 20%, 23% and 25% (and ranges in between). In one embodiment, when decreased fucosylation is desired, the cell can be cultured at CO₂ levels of about 11 to 25%, e.g., about 15%. CO₂ levels can be adjusted manually or can be a cell byproduct.

A wide array of flasks, bottles, reactors, and controllers allow the production and scale up of cell culture systems. The system can be chosen based, at least in part, upon its correlation with a desired glycan property or properties.

Cells can be grown, for example, as batch, fed-batch, perfusion, or continuous cultures.

Production parameters that can be selected include, e.g., addition or removal of media including when (early, middle or late during culture time) and how often media is harvested; increasing or decreasing speed at which cell cultures are agitated; increasing or decreasing temperature at which cells are cultured; adding or removing media such that culture density is adjusted; selecting a time at which cell cultures are started or stopped; and selecting a time at which cell culture parameters are changed. Such parameters can be selected for any of the batch, fed-batch, perfusion and continuous culture conditions.

### Glycoproteins

Subject glycoproteins include naturally occurring and normaturally occurring glycoproteins. Representative glycoproteins include: antibodies, e.g., IgG, IgM, human, humanized, grafted, and chimeric antibodies, and fragments thereof; fusion proteins, e.g., fusions including human (or other) antibody domains, e.g., Fc or constant region domains; growth factors; hormones, interferons; cytokines; cytokine receptors; soluble blood components, e.g., albumin, clotting factors, hematopoietic factors; enzymes; and any class of protein represented by a protein listed in Table 3. Also included are soluble or active fragments of any of the glycoprotiens or classes of glycoprotein discussed herein.

Exemplary glycoproteins that can be made by methods described herein include those in Table 3 below.

TABLE 3

| Protein Product | Reference Listed Drug |
|---|---|
| interferon gamma-1b | Actimmune ® |
| alteplase; tissue plasminogen | Activase ®/Cathflo ® |
| activator | |
| Recombinant antihemophilic | Advate |
| factor | |
| human albumin | Albutein ® |
| Laronidase | Aldurazyme ® |
| interferon alfa-N3, human | Alferon N ® |
| leukocyte derived | |
| human antihemophilic factor | Alphanate ® |
| virus-filtered human coagulation | AlphaNine ® SD |
| factor IX | |
| Alefacept; recombinant, dimeric | Amevive ® |
| fusion protein LFA3-Ig | |
| Bivalirudin | Angiomax ® |
| darbepoetin alfa | Aranesp ™ |
| Bevacizumab | Avastin ™ |
| interferon beta-1a; recombinant | Avonex ® |
| coagulation factor IX | BeneFix ™ |
| Interferon beta-1b | Betaseron ® |
| Tositumomab | BEXXAR ® |
| antihemophilic factor | Bioclate ™ |
| human growth hormone | BioTropin ™ |
| botulinum toxin type A | BOTOX ® |
| Alemtuzumab | Campath ® |
| acritumomab; technetium-99 | CEA-Scan ® |
| labeled | |
| alglucerase; modified form of | Ceredase ® |
| beta-glucocerebrosidase | |
| imiglucerase; recombinant form | Cerezyme ® |
| of beta-glucocerebrosidase | |
| crotalidae polyvalent immune | CroFab ™ |
| Fab, ovine | |
| digoxin immune | DigiFab ™ |
| fab [ovine] | |
| Rasburicase | Elitek ® |
| Etanercept | ENBREL ® |
| epoietin alfa | Epogen ® |
| Cetuximab | Erbitux ™ |
| algasidase beta | Fabrazyme ® |
| Urofollitropin | Fertinex ™ |
| follitropin beta | Follistim ™ |
| Teriparatide | FORTEO ® |
| human somatropin | GenoTropin ® |
| Glucagon | GlucaGen ® |
| follitropin alfa | Gonal-F ® |
| antihemophilic factor | Helixate ® |
| Antihemophilic Factor; | HEMOFIL |
| Factor XIII | |
| adefovir dipivoxil | Hepsera ™ |
| Trastuzumab | Herceptin ® |
| Insulin | Humalog ® |
| antihemophilic factor/von | Humate-P ® |
| Willebrand factor | |
| complex-human | |
| Somatotropin | Humatrope ® |
| Adalimumab | HUMIRA ™ |
| human insulin | Humulin ® |
| recombinant human | Hylenex ™ |
| hyaluronidase | |
| interferon alfacon-1 | Infergen ® |
| eptifibatide | Integrilin ™ |
| alpha-interferon | Intron A ® |
| Palifermin | Kepivance |
| Anakinra | Kineret ™ |
| antihemophilic factor | Kogenate ® FS |
| insulin glargine | Lantus ® |
| granulocyte macrophage | Leukine ®/Leukine ® Liquid |
| colony-stimulating factor | |
| lutropin alfa for injection | Luveris |
| OspA lipoprotein | LYMErix ™ |
| Ranibizumab | LUCENTIS ® |
| gemtuzumab ozogamicin | Mylotarg ™ |
| Galsulfase | Naglazyme ™ |
| Nesiritide | Natrecor ® |
| Pegfilgrastim | Neulasta ™ |
| Oprelvekin | Neumega ® |
| Filgrastim | Neupogen ® |
| Fanolesomab | NeutroSpec ™ (formerly LeuTech ®) |
| somatropin [rDNA] | Norditropin ®/Norditropin Nordiflex ® |
| Mitoxantrone | Novantrone ® |
| insulin; zinc suspension; | Novolin L ® |
| insulin; isophane suspension | Novolin N ® |
| insulin, regular; | Novolin R ® |
| Insulin | Novolin ® |
| coagulation factor VIIa | NovoSeven ® |
| Somatropin | Nutropin ® |
| immunoglobulin intravenous | Octagam ® |
| PEG-L-asparaginase | Oncaspar ® |
| abatacept, fully human soluable | Orencia ™ |
| fusion protein | |
| muromomab-CD3 | Orthoclone OKT3 ® |
| high-molecular weight | Orthovisc ® |
| hyaluronan | |
| human chorionic gonadotropin | Ovidrel ® |
| live attenuated Bacillus | Pacis ® |
| Calmette-Guerin | |
| peginterferon alfa-2a | Pegasys ® |
| pegylated version of interferon | PEG-Intron ™ |
| alfa-2b | |
| Abarelix (injectable suspension); | Plenaxis ™ |
| gonadotropin-releasing hormone | |
| antagonist | |
| epoietin alfa | Procrit ® |
| Aldesleukin | Proleukin, IL-2 ® |
| Somatrem | Protropin ® |
| dornase alfa | Pulmozyme ® |
| Efalizumab; selective, | RAPTIVA ™ |
| reversible T-cell blocker | |
| combination of ribavirin and | Rebetron ™ |
| alpha interferon | |
| Interferon beta 1a | Rebif ® |
| antihemophilic factor | Recombinate ® rAHF/ |
| antihemophilic factor | ReFacto ® |
| Lepirudin | Refludan ® |
| Infliximab | REMICADE ® |
| Abciximab | ReoPro ™ |
| Reteplase | Retavase ™ |
| Rituxima | Rituxan ™ |
| interferon alfa-2^{a} | Roferon-A ® |
| Somatropin | Saizen ® |
| synthetic porcine secretin | SecreFlo ™ |
| Basiliximab | Simulect ® |
| Eculizumab | SOLIRIS (R) |
| Pegvisomant | SOMAVERT ® |
| Palivizumab; recombinantly | Synagis ™ |
| produced, humanized mAb | |
| thyrotropin alfa | Thyrogen ® |
| Tenecteplase | TNKase ™ |
| Natalizumab | TYSABRI ® |
| human immune globulin | Venoglobulin-S ® |
| intravenous 5% and | |
| 10% solutions | |
| interferon alfa-n1, | Wellferon ® |
| lymphoblastoid | |
| drotrecogin alfa | Xigris ™ |
| Omalizumab; recombinant | Xolair ® |
| DNA-derived humanized | |
| monoclonal antibody | |
| targeting immunoglobulin-E | |
| Daclizumab | Zenapax ® |
| ibritumomab tiuxetan | Zevalin ™ |
| Somatotropin | Zorbtive ™ (Serostim ®) |

In some embodiments, the method described herein can be used to make glycoproteins having a selected level of high mannose, e.g., an increased level of high mannose, as compared to a reference glycoprotein.

### EXAMPLES

### Example 1

### Analysis of CTLA4 Glycans Produced in Various Isolates and Use of Glycan Data to Distinguish Cells from Different Backgrounds

Four CHO cell line backgrounds were transfected with the gene encoding for CTLA4IgG. These pools of cells were then subjected to selection and clonal selection to generate 20 clones from each of the four cell line backgrounds. CTLA4IgG produced from each clone was isolated and purified by protein A affinity chromatography. The glycans from the isolated glycoprotein were then released, labeled and analyzed by LC and LC/MS. An illustrative chromatogram is described in FIG. 1. Illustrative LC data of the distribution of the product from each of the clones is described in FIG. 2.

Analysis of glycan data was used to distinguish cells from the four backgrounds. Data on a number of aspects of glycan structure was determined. Representative aspects of glycan structure which can be used in this approach are provided in Table 4. In this table glycans are represented as the composition of HexNAc, Hex, Fuc, NeuAc, NeuGc, the presence of an A, or B indicates the isomeric species and the presence of Ac indicates an acetylation event.

TABLE 4

| | | |
|---|---|---|
| 4, 3, 1, 0, 0 | 4, 5, 1, 1, 0 + 2Ac | 4, 5, 1, 2, 0 |
| 3, 4, 1, 0, 0 | 4, 4, 1, 1, 0 | 5, 6, 1, 2, 0 |
| 4, 4, 1, 0, 0 A | 4, 5, 1, 1, 0 A | 6, 6, 1, 2, 0 |
| 4, 4, 1, 0, 0B | 4, 5, 1, 1, 0 B | 6, 7, 1, 2, 0 |
| 4, 5, 1, 0, 0 | 6, 6, 1, 1, 0 | 7, 8, 1, 2, 0 |
| 5, 5, 1, 0, 0 | 6, 7, 1, 1, 0 | 5, 6, 1, 3, 0 + 2Ac |
| 4, 6, 1, 0, 0 | 4, 5, 1, 2, 0 + 4Ac | 5, 6, 1, 3, 0 + Ac |
| 5, 6, 1, 0, 0 | 4, 5, 1, 2, 0 + 3Ac | 6, 7, 1, 3, 0 + 2Ac |
| 4, 4, 1, 1, 0 + 2Ac | 4, 5, 1, 2, 0 + 2Ac | 5, 6, 1, 3, 0 A |
| 4, 4, 1, 1, 0 + Ac | 4, 5, 1, 2, 0 + Ac | 5, 6, 1, 3, 0 B |
| 6, 7, 1, 3, 0 A + Ac | 6, 7, 1, 3, 0 | 4, 5, 1, 2, 0 + SO3 + |
| | | Ac |
| 6, 7, 1, 3, 0 B + Ac | 4, 5, 1, 2, 0 + SO3 + 2Ac | 4, 5, 1, 2, 0 + SO3 |
| 5, 6, 1, 2, 0 + SO3 | 6, 7, 1, 4, 0 + 2Ac | 6, 7, 1, 4, 0 + Ac |
| 6, 7, 1, 4, 0 | | |

The glycan data were then subjected to Principal Component Analysis (PCA). PCA provided the plot shown in FIG. 3. Although easier imaged as a rotating 3 dimensional image, angle 86 was chose as a representative image of the PCA analysis as it best illustrates in 2 dimensions the distribution of the clones. Surprisingly, this analysis provides a robust differentiation between members of this group of relatively similar cell types. Surprisingly the cell population quality attribute profiles for each of the cell types, CHO K1, CHO S, CHO DG44 and DHfr(−) are not only distinct but allow unambiguous selection of a cell line having a desired quality, e.g., as shown by the differentiation along the X axis.

### Example 2

### Correlation of Glycan Structures with Gene Expression Data Using Linear Models

Four Chinese Hamster Ovary cell lines were transfected with a gene to produce CTLA4-Ig protein. Clones from each cell line were obtained by dilution cloning; clonal cell lines were expanded in order to produce CTLA4-Ig protein for glycans analysis and RNA for gene expression analysis. Cellular RNA and CTLA4-Ig protein were obtained from 20-24 clones from each cell line. Messenger RNA (mRNA) was analyzed by RT-PCR to measure the expression levels of 28 glycosylation-related genes. Expression levels of glycosylation-related were normalized by one or more housekeeping genes (i.e. β-actin or ribosomal protein genes). Linearized expression levels were obtained by exponential transformation of the housekeeping-gene normalized expression level. These data are illustrated in FIG. 4. Glycans were obtained from the CTLA4-Ig protein and analyzed by several methods including LC MS/MS. Percent composition was calculated for each glycan species. Representative data are shown in FIG. 5.

Linear modeling was employed to discover relationships between glycans structure and gene expression. Linear model discovery was performed with the software environment R using the following method. For each measured glycan the dataset was divided into training and test sets using a bootstrap with stratification method to ensure equal representation of isolates from the four cell lines. The best fit coefficients of the linear model for each individual gene were computed and recorded for the training set; model fit error was recorded. Gene expression levels were used to calculate the glycan level for samples in the test set; estimation error was recorded. The linear model with best fit to the training set was retained. All two-gene models were evaluated by adding in turn each remaining gene to the best fit one-gene model. The best fit two-gene model was retained. This process was repeated until models of 10-15 genes were generated. The entire process was repeated from generation of training and test sets for 20 iterations for each glycan in order to measure repeatability of the discovery of best fit models.

Detailed model analysis was subsequently performed. Models utilizing more than 5 genes were determined to be undesirable due to universally high error rates for test sets which indicates overfitting of the data. For each glycan, the frequency of occurrence was computed of a particular gene in the first five positions of the 20 model discovery runs. The most frequently occurring genes were selected for detailed modeling analysis in which 200 iterations of training and test set error rates were computed using bootstrap with stratification followed by coefficient computation for the best fit linear model employing the target genes. Error was recorded for training and test sets for each iteration. Models with desirable training and test errors were subsequently compared to each other by fitting the model to the entire data set performing F-tests of model errors to justify the selection of more complex models over simple models.

In the example included here (see FIG. 6), a linear model utilizing ST3GAL3 expression to compute the level of glycan G5.6.1.2.0 produced a reasonable fit to the measured level of the glycan. A linear model with ST3GAL4 did not produce a model with adequate fit. However addition of ST3GAL4 to the ST3GAL3 model produced a model with a significantly better fitting model to the data according to F-test (p=0.0011). The sign of the coefficients for the two genes indicate that increased expression of ST3GAL3 increases the level of G5.6.1.2.0 and increased expression of ST3GAL4 decreases the level of G5.6.1.2.0. This relationship was unexpected.

| | (Intercept) | ST3GAL3 | Train.Rsq | Test.Rsq | (Intercept) | ST3GAL3 | ST3GAL4 | Train.Rsq | Test.Rsq |
|---|---|---|---|---|---|---|---|---|---|
| mean | 0.22 | 226.68 | 0.58 | 0.48 | 1.02 | 272.25 | −51.21 | 0.72 | 0.60 |
| median | 0.23 | 223.96 | 0.57 | 0.49 | 1.01 | 267.61 | −49.76 | 0.71 | 0.62 |
| sd | 0.30 | 27.16 | 0.10 | 0.17 | 0.33 | 27.84 | 12.75 | 0.08 | 0.17 |

Analysis of Variance Table
Model 1: G5.6.1.2.0~ST3GAL3
Model 2: G5.6.1.2.0~ST3GAL3 + ST3GAL4
Res.Df RSS Df Sum of Sq F Pr(>F)
1 29 30.5688
2 28 20.7739 1 9.7949 13.202 0.001112 **

### Example 3

### Cell Line Variability and Classification

Four Chinese Hamster Ovary cell lines were transfected with a gene to produce CTLA4-Ig protein. Clones from each cell line were obtained by dilution cloning; clonal cell lines were expanded in order to produce CTLA4-Ig protein for glycans analysis and RNA for gene expression analysis. Cellular RNA and CTLA4-Ig protein were obtained from 20-24 clones from each cell line. Messenger RNA (mRNA) was analyzed by RT-PCR to measure the expression levels of 28 glycosylation-related genes. Expression levels of glycosylation-related were normalized by one or more housekeeping genes (i.e. β-actin or ribosomal protein genes). Linearized expression levels were obtained by exponential transformation of the housekeeping-gene normalized expression level.

Transcriptional data profiles for a variety of genes related to glycosylation are illustrated in FIG. 7. FIG. 7 depicts the distribution of transcripts related to glycosylation across the clones (each dot) from each cell line background (Blue, Red, Green, or Black) clustered for each transcript. The genes followed were as follows: transcripts A1-A8, B1-5, C5,6 are from glycosyltransferases; B6-8, C1-4, D1-4, are from biosynthetic enzymes; C7,8, D5,6, are normalizing and CTLA4IgG transcripts. The transcriptional data was then subjected to Principal Component Analysis (PCA) blinded to the cell line background ID. The first three principal components were plotted on x-, y-, and z-axes. The clones were then ascribed a symbol according to their cell line origin as illustrated in FIG. 8. Surprisingly the cell population quality attribute profiles for each of the cell types, CHO K1, CHO S, CHO DG44 and DHfr(−) are not only distinct but allow unambiguous selection of a cell line having a desired quality, e.g., as shown by the views in FIG. 8.

A blind assay was then conducted in which the transcriptional profile was measured for 21 cell isolates of unknown origin. The origin of each cell line was blinded to the experimenters. However, they were known to have the potential to be derived from any of the CHO cell lines K1, S, DG44 and DHfr(−). The data from the isolates of unknown origin was transformed into the coordinate system used in the PCA of the original data and plotted along with the original data. See FIG. 9, which shows the unknowns superimposed on the cell population quality attribute profiles for each of the four cell types derived from known origins. The identity of each cell line was predicted by linear discriminant analysis (LDA); 20 out of 21 clones were correctly classified. The cell population quality attribute profiles allowed correct assignment of one of the four cell types to 20 out of the 21 unknown cell isolates.

### Extensions and Alternatives

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way. While the methods have been described in conjunction with various embodiments and examples, it is not intended that the methods be limited to such embodiments or examples. On the contrary, the methods encompass various alternatives, modifications, and equivalents, as will be appreciated by those of skill in the art.

1. A method of making a glycoprotein having a selected glycan complement or glycan component comprising:
(a) acquiring the identity of a cell population for the production of said glycoprotein, wherein the identity is acquired or determined by
(i) acquiring, for each of a plurality of isolates or aliquots of a first cell population, a value which is expressed in terms of a glycan complement or glycan component, which value is a function of a plurality of distinct observations that include the level of expression of a plurality of different genes and the level of expression of a plurality of different glycostructures, glycan structures, glycan components, or combinations thereof to provide a set of values for said first cell population;
(ii) acquiring, for each of a plurality of isolates or aliquots of a second cell population, a value which is expressed in terms of a glycan complement or glycan component, which value is a function of a plurality of distinct observations that include the level of expression of a plurality of different genes and the level of expression of a plurality of different glycostructures, glycan structures, glycan components, or combinations thereof to provide a set of values for said second cell population;
(iii) comparing a value for a selected glycan complement or glycan component with the set of values for said first cell population and with the set of values for said second cell population; and
(iv) responsive to said comparison, selecting said first or second cell population; and
(b) culturing said selected cell population, to thereby make said glycoprotein having said selected glycan complement or glycan component.

2. The method of claim 1, further comprising isolating said glycoprotein from the culture.

3. The method of claim 2, further comprising purifying said glycoprotein.

4. The method of claim 1, further comprising
(i) acquiring a cell population quality attribute profile (a profile), comprising a set of answers, wherein an answer is expressed in terms of a glycan complement or glycan component and is the product of an operation on a plurality of observations, for each of a plurality of cell populations, said acquired profiles forming a plurality of distinct profiles;
(ii) acquiring the identity of a selected glycan complement or glycan component;
(iii) comparing the acquired profile with the identity acquired in (ii);
(iv) when the acquired profile includes the identity acquired in (ii), selecting one of the plurality of cell populations to make said glycoprotein having the selected glycan complement or glycan component.

5. The method of claim 1, further comprising introducing a nucleic acid that encodes all or part of said glycoprotein into said identified cell population.

6. The method of claim 1, wherein the first cell population is a CHO cell population and the second cell population is a second CHO cell population, wherein said second CHO cell population differs from said first CHO cell population by a naturally acquired or intentionally induced mutation.

7. The method of claim 6, wherein the observation is one or more of the level of 4,4,1,0,0; the level of 4,4,1,1,0; the level of 4,5,1,0,0; the level of 4,5,1,1,0; the level of 4,5,1,2,0; the level of 5,5,1,0,0; the level of 5,6,1,0,0; the level of 5,6,1,1,0; the level of 5,6,1,2,0; the level of 5,6,1,3,0; the level of 6,6,1,1,0; the level of 6,6,1,2,0; the level of 6,7,1,1,0; the level of 6,7,1,2,0; the level of 6,7,1,3,0; the level of 6,7,1,4,0; the level of expression of a glycosyltransferase; the level of expression of a gene involved in glycan biosynthesis; the level of a metabolite; the level of UMP; the level of GTP; the level of UDP-Gal; the level of GDP-Fuc.

8. The method of claim 1, wherein a set of values is acquired for a plurality of CHO cell populations including a CHO K1 cell line, a CHO S cell line, a DG44 cell line and a DHFR(−) cell line.

9. A method of providing or selecting a cell population from a plurality of isolates from a cell population for use in making a glycoprotein having a selected glycan complement or glycan component, comprising:
(a) acquiring the identity of a selected glycan complement or glycan component;
(b) acquiring an evaluation of the ability of each of said plurality of isolates of said cell population to produce said glycan complement or glycan component, and
(c) selecting an isolate from said plurality of isolates, to thereby provide an isolate from a cell population for use in making a glycoprotein having a selected glycan complement or glycan component.

10. A method of monitoring a production process for making a glycoprotein having a selected post-translational modification, comprising:
(a) acquiring, for each of for each of a plurality of isolates or aliquots of a first cell population, a value which is expressed in terms of a glycan complement or glycan component, which value is a function of a plurality of distinct observations that include the level of expression of a plurality of different genes and the level of expression of a plurality of different glycostructure, glycan complement or glycan component to provide a set of values for said first cell population;
(b) identifying a selected glycan complement or glycan component;
(c) comparing a value for the selected glycan complement or glycan component, with the set of values for said first cell population; and
(d) if the comparison shows that the set of values for said first cell population includes the value for the selected glycan complement or glycan component, pursuing a first option, e.g., continuing culture; and if the comparison shows that the set of values does not include the value for the selected glycan complement or glycan component, pursuing a second option, e.g., ceasing current culture conditions or culturing under a new set of conditions.

11. A method of selecting a glycoprotein for manufacture in a cell population, comprising:
(a) acquiring a cell population quality attribute profile, comprising a set of answers, wherein an answer is expressed in terms of a glycan complement or glycan component and is the product of an operation on a plurality of observations, for a cell population;
(b) acquiring the identities of a plurality of glycan complement or glycan component;
(c) comparing the acquired profile with the identities acquired in (b);
(d) when the identities acquired in (b) include the acquired profile, selecting one of the plurality glycan complement or glycan component for production in said cell population; and
(e) making a glycoprotein having the selected glycostructure in said cell population.

12. A data base comprising a plurality of records for isolates of a cell population of a preselected cell population, wherein each record comprises an identifier for a unique isolate of said preselected cell type and an identifier for a cell population quality attribute profile unique for the isolate, and wherein said cell population quality attribute profile for each entry is unique as opposed to others in the plurality for the isolate.

13. The method of claim 1, wherein the selected glycan component is a high mannose structure.

14. The method of claim 1, wherein the observations for each cell population include at least 2, 3, 4, 5, 6, 7, 8, 9, 10 or more observations of the expression level of genes.

15. The method of claim 1, wherein the glycoprotein is a therapeutic biologic product.

16. The method of claim 1, wherein the glycoprotein is a biosimilar or biogeneric version of a marketed biologic product.
